(19) United States
(12) Patent Application Publication
(10) Pub. No.: US 2007/0196453 A1
(43) Pub. Date: Aug. 23, 2007

(54) Two or more non-volatile solvent-containing compositions and methods for dermal delivery of drugs

(76) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,445

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/577,536, filed on Jun. 7, 2004.

(51) Int. Cl. A61K 9/70

(52) U.S. Cl. 424/443

(57) ABSTRACT

The present invention is drawn to adhesive formulations, methods of drug delivery, and solidified layers for dermal delivery of a drug. The formulation can include a drug, a solvent vehicle, and a solidifying agent. The solvent vehicle can have a volatile solvent system including at least one volatile solvent, and a non-volatile solvent system including at least two non-volatile solvents. The formulation can have a viscosity suitable for application to a skin surface prior to evaporation of the volatile solvents system. When applied to the skin, the formulation can form a solidified layer after at least a portion of the volatile solvent system is evaporated.

This application claims the benefit of U.S. Provisional Application No. 60/750,637, which was filed on Dec. 14, 2005, and is a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to systems developed for dermal delivery of drugs. More particularly, the present invention relates to formulations including at least two non-volatile solvents, wherein the formulation as a whole has a viscosity suitable for application as a layer to a skin surface, and which forms a sustained drug-delivering adhesive solidified layer on the skin.

### BACKGROUND OF THE INVENTION

Traditional dermal drug delivery systems can generally be classified into two forms: semisolid formulations and dermal patch dosage forms. Semisolid formulations are available in a few different forms, including ointments, creams, foams, pastes, gels, or lotions and are applied topically to the skin. Dermal (including transdermal) patch dosage forms also are available in a few different forms, including matrix patch configurations and liquid reservoir patch configurations. In a matrix patch, the active drug is mixed in an adhesive that is coated on a backing film. The drug-laced adhesive layer is typically directly applied onto the skin and serves both as means for affixing the patch to the skin and as a reservoir or vehicle for facilitating delivery of the drug. Conversely, in a liquid reservoir patch, the drug is typically incorporated into a solvent system which is held by a thin bag, which can be a thin flexible container. The thin bag can include a permeable or semi-permeable membrane surface that is coated with an adhesive for affixing the membrane to the skin. The membrane is often referred to as a rate limiting membrane (although it may not actually be rate limiting in the delivery process in all cases) and can control transport of the drug from within the thin bag to the skin for dermal delivery.

While patches and semisolid formulations are widely used to deliver drugs into and through the skin, they both have significant limitations. For example, most semisolid formulations usually contain solvent(s), such as water and ethanol, which are volatile and thus evaporate shortly after application. The evaporation of such solvents can cause a significant decrease or even termination of dermal drug delivery, which may not be desirable in many cases. Additionally, semisolid formulations are often “rubbed into” the skin, which does not necessarily mean the drug formulation is actually delivered-into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing. Drugs present in a semisolid formulation may also be unintentionally delivered to persons who come in contact with a subject undergoing treatment with a topical semisolid formulation.

With respect to matrix patches, in order to be delivered appropriately, a drug should have sufficient solubility in the adhesive, as primarily only dissolved drug contributes to the driving force required for skin permeation. Unfortunately, solubility in adhesives that is too low does not generate adequate skin permeation driving force over sustained period of time. In addition, many ingredients, e.g., liquid solvents and permeation enhancers, which could be used to help dissolve the drug or increase the skin permeability, may not be able to be incorporated into many adhesive matrix systems in sufficient quantities to be effective. For example, at functional levels, most of these materials may adversely alter the wear properties of the adhesive. As such, the selection and allowable quantities of additives, enhancers, excipients, or the like in adhesive-based matrix patches can be limited. To illustrate, for many drugs, optimal transdermal flux can be achieved when the drug is dissolved in certain liquid solvent systems, but a thin layer of adhesive in a typical matrix patch often cannot hold enough appropriate drug and/or additives to be therapeutically effective. Further, the properties of the adhesives, such as coherence and tackiness, can also be significantly changed by the presence of liquid solvents or enhancers.

Regarding liquid reservoir patches, even if a drug is compatible with a particular liquid or semisolid solvent system carried by the thin bag of the patch, the solvent system still has to be compatible to the adhesive layer coated on the permeable or semi-permeable membrane; otherwise the drug may be adversely affected by the adhesive layer or the drug/solvent system may reduce the tackiness of the adhesive layer. In addition to these dosage form considerations, reservoir patches are bulkier and usually are more expensive to manufacture than matrix patches.

Another shortcoming of dermal (including transdermal) patches is that they are usually neither stretchable nor flexible, as the backing film (in matrix patches) and the thin fluid bag (in reservoir patches) are typically made of polyethylene or polyester, both of which are relatively non-stretchable materials. If the patch is applied to a skin area that is significantly stretched during body movements, such as a joint, separation between the patch and skin may occur thereby compromising the delivery of the drug. In addition, a patch present on a skin surface may hinder the expansion of the skin during body movements and cause discomfort. For these additional reasons, patches are not ideal dosage forms for skin areas subject to expansion, flexing and stretching during body movements.

In view of the shortcomings of many of the current delivery systems, it would be desirable to provide systems, formulations, and/or methods that can i) provide sustained drug delivery over long periods of time; ii) are not vulnerable to unintentional removal by contact with clothing, other objects, or people for the duration of the application time; iii) can be applied to a skin area subject to stretching and expansion without causing discomfort or poor contact to skin; and/or iv) can be easily removed after application and use.

### SUMMARY OF THE INVENTION

Although film-forming technologies have been used in cosmetic and pharmaceutical preparations, typically, the solvents used in such systems evaporate shortly after application, and thus, are not optimal for sustained-release applications. In accordance with this, it has been recognized that the use of multiple non-volatile solvents in the formulation can often optimize sustained drug delivery.

In accordance with this, it would be advantageous to provide dermal delivery formulations, systems, and/or methods in the form of adhesive compositions or formulations having a viscosity suitable for application to the skin surface and which form a drug-delivering solidified layer on the skin that is optionally peelable or otherwise easily removable after use. As such, an adhesive formulation for dermal delivery of a drug can comprise a drug, a solvent vehicle, and a solidifying agent. The solvent vehicle can comprise a volatile solvent system including at least one volatile solvent and a non-volatile solvent system including at least two non-volatile solvents. The at least two non-volatile solvents of the non-volatile solvent system can facilitate transdermal delivery of the drug at a therapeutically effective rate over a sustained period of time, even after the non-volatile solvent system is substantially evaporated from the solidified layer. The formulation can have viscosity suitable for application to the skin surface prior to evaporation of at least one volatile solvent, and can further be formulated such that when applied to the skin surface, the formulation forms a solidified layer after at least a portion of the volatile solvent system is evaporated. Sustained drug delivery from the solidified layer can also occur.

In an alternative embodiment, a method of dermally delivering a drug can comprise applying an adhesive formulation to a skin surface of a subject. The formulation can comprise a drug, solvent vehicle, and a solidifying agent. The solvent vehicle can comprise a volatile solvent system including at least one volatile solvent, and a non-volatile solvent system including at least two non-volatile solvents, wherein the non-volatile solvent system facilitates dermal delivery of the drug at a therapeutically effective rate over a sustained period of time. The formulation can have a viscosity suitable for application and adhesion to the skin surface prior to evaporation of the volatile solvent system. Other steps include solidifying the formulation to form a solidified layer on the skin surface by at least partial evaporation of the volatile solvent system, and dermally delivering the drug from the solidified layer to the skin surface at therapeutically effective rates over a sustained period of time.

In another embodiment, a solidified layer for delivering a drug can comprise a drug, a non-volatile solvent system including at least two non-volatile solvents, wherein the non-volatile solvent system is capable of facilitating the delivery of the drug at therapeutically effective rates over a sustained period of time, and a solidifying agent. In one embodiment, the solidified layer can be stretchable by 5% in one direction without cracking, breaking, or separating from a skin surface to which the layer is applied.

Additional features and advantages of the invention will be apparent from the following detailed description and figures which illustrate, by way of example, features of the invention.

### BRIEF DESCRIPTION OF THE DRAWINGS

FIG. 1 is a graphical representation of cumulative amount of testosterone delivered across a biological membrane in vitro over time from a solidified adhesive formulation and a marketed product (AndroGel) in accordance with embodiments of the present invention.

FIG. 2 is a graphical representation of the cumulative amount of acyclovir delivered transdermally over time from two separate formulations in accordance with embodiments of the present invention compared to the marketed product Zovirax cream.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

The term “drug(s)” refers to any bioactive agent that is applied to, into, or through the skin which is applied for achieving a therapeutic affect. This includes compositions that are traditionally identified as drugs, as well other bioactive agents that are not always considered to be “drugs” in the classic sense, e.g., peroxides, humectants, emollients, etc., but which can provide a therapeutic effect for certain conditions. When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced herein can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic effective levels of the drug across, onto or into the subject's skin when the non-volatile solvent system is saturated with the drug. For systemically targeted drugs, a flux enabling non-volatile solvent system is a non-volatile solvent system that can provide therapeutically effective daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically effective amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide effective drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” is defined as the permeation flux of the selected drug that delivers sufficient amount of drug into or across the skin to be clinically beneficial in that some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time.

Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400 cm²/10 hour, which equals 1 mcg/cm²/hr. By this definition, different drugs have different “therapeutically effective flux. Therapeutically effective flux may also be different in different subjects and or at different times for even the same subject. However, for each drug, there is usually a consensus among the skilled in the art on the range of doses or fluxes that are sufficient in most subjects at most times.

The following are estimates of flux for some drugs that are therapeutically effective:

TABLE 1
In vitro steady state flux values of various drugs

| | | Estimated Therapeutically |
|---|---|---|
| | | effective flux* |
| Drug | Indication | (mcg/cm²/h) |
| Ropivacaine** | Neuropathic pain | 5 |
| Lidocaine | Neuropathic pain | 30 |
| Acyclovir | Herpes simplex virus | 3 |
| Ketoprofen | Musculoskeletal pain | 16 |
| Diclofenac | Musculoskeletal pain | 1 |
| Clobetasol | Dermatitis, psoriasis, | 0.05 |
| | eczema | |
| Betamethasone | Dermatitis, psoriasis, | 0.01 |
| | eczema | |
| Testosterone | Hypogonadal men, | 0.8 |
| Testosterone | Hormone treatment for | 0.25 |
| | postmenopausal women | |
| Imiquimod | Warts, basal cell | 0.92 |
| | carcinoma | |

*Flux determined using an in vitro method described in Example 1.
**Estimated flux based on known potency relative to lidocaine.

The therapeutically effective flux values in Table 1 (with the exception of ropivacaine) represent the steady state flux values of marketed products through hairless mouse or human epidermal membrane in an in vitro system described in Example 1. These values are meant only to be estimates and to provide a basis of comparison for formulation development and optimization. The therapeutically effective flux for a selected drug could be very different for different diseases to be treated for, different stages of diseases, different individual subjects, etc. It should be noted that the flux listed may be more than therapeutically effective.

The following examples listed in Table 2 illustrate screening of a non-volatile solvent's flux enabling ability for some of the drugs specifically studied. Experiments were carried out as described in Example 1 below and the results are further discussed in the subsequent Examples 2-9.

TABLE 2
In vitro steady state flux values of various
drugs from non-volatile solvent systems

| | | Average Flux* |
|---|---|---|
| Drug | Non-Volatile Solvent | (mcg/cm²/hr) |
| Betamethasone | Oleic acid | 0.009 ± 0.003 |
| Dipropionate | Sorbitan Monolaurate | 0.03 ± 0.02 |
| Clobetasol Propionate | Propylene Glycol (PG) | 0.0038 ± 0.0004 |
| | Light Mineral Oil | 0.031 ± 0.003 |
| | Isostearic acid (ISA) | 0.019 ± 0.003 |
| Ropivacaine | Glycerol | 1.2 ± 0.7 |
| | Mineral Oil | 8.9 ± 0.6 |
| Ketoprofen | Polyethylene glycol 400 | 5 ± 2 |
| | Span 20 | 15 ± 3 |
| Acyclovir | Polyethylene glycol 400 | 0 |
| | Isostearic acid + 10% | 2.7 ± 0.6 |
| | trolamine | |

*Each value represents the mean and st. dev of three determinations.

The in vitro steady state flux values in Table 2 from non-volatile solvents show surprising flux-enabling and non flux-enabling solvents. This information can be used to guide formulation development.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

Different drugs often have different matching flux-enabling non-volatile solvent systems which provide particularly good results. Examples of such are noted in Table 3. Experiments were carried out as described in Example 1 below.

TABLE 3
In vitro steady state flux values of various drugs from particularly
high flux-enabling non-volatile solvent systems

| | High flux-enabling non- | Avg. Flux* |
|---|---|---|
| Drug | volatile solvent | (mcg/cm²/h) |
| ropivacaine | ISA | 11 ± 2 |
| | Span 20 | 26 ± 8 |
| ketoprofen | Propylene glycol (PG) | 90 ± 50 |
| acycolvir | ISA + 30% trolamine | 7 ± 2 |
| Betamethasone | Propylene Glycol | 0.20 ± 0.07 |
| Dipropionate | | |
| Clobetasol | PG + ISA (Ratio of PG:ISA | 0.8 ± 0.2 |
| propionate | ranging from 200:1 to 1:1) | |

*Each value represents the mean and st. dev of three determinations.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table 3 is a 9:1 for propylene glycol:isostearic acid mixture that generated much higher clobetasol flux than propylene glycol or ISA alone (see Table 2). Therefore, the 9:1 propylene glycol:isostearic acid mixture is a “high flux-enabling non-volatile solvent” but propylene glycol or isostearic acid alone is not.

The term “adhesion” or “adhesive” when referring to a solidified layer herein refers to sufficient adhesion between the solidified layer and the skin so that the layer does not fall off the skin during intended use on most subjects. Thus, “adhesive” or the like when used to describe the solidified layer means the solidified layer is adhesive to the skin surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the skin surface. For example, excessively sweating or oily skin, or oily substances on the skin surface may make the solidified layer less adhesive to the skin. Therefore, the adhesive solidified layer of the current invention may not be able to maintain perfect contact with the skin surface and deliver the drug over a sustained period of time for every subject under any conditions on the skin surface. A standard is that it maintains good contact with most of the skin surface, e.g. 70% of the total area, over the specified period of time for most subjects under normal conditions of the skin surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” in this invention is defined as a mixture of at least two solvents that are each less volatile than water. Similarly, a non-volatile solvent is defined as a solvent that is less volatile than water. The non-volatile solvent system can also contain substances that are solid or liquid at room temperatures, such as pH or ion-pairing agents. After evaporation of the volatile solvent system, most of the non-volatile solvent system should remain in the solidified layer for a period of time sufficient to adequately dermally deliver a given drug to, into, or through the skin of a subject at a sufficient flux for a period of time to provide a therapeutic effect.

The non-volatile solvent system can also serve as a plasticizer of the solidified layer, so that the solidified layer is elastic and flexible. In one embodiment, the non-volatile solvent system provides better plasticizing effects for the solidifying agents than any single non-volatile solvent of the non-volatile solvent system alone. Including multiple non-volatile solvents as part of the non-volatile solvent system can also provide various other benefits. In some cases, a single non-volatile solvent may not provide the formulation with adequate compatibility with other ingredients in the formulation, e.g. volatile solvent system or solidifying agent, and/or the ability to generate therapeutically effective flux of the drug. In one aspect of the invention, the non-volatile solvent system provides better compatibility with the solidifying agent than any single non-volatile solvent of the non-volatile solvent system alone. In another aspect of the invention, the non-volatile solvent system provides higher flux than any single non-volatile solvent of the non-volatile solvent system alone. The present invention allows for combinations of two or more non-volatile solvents which together are able to provide both therapeutically effective drug flux while maintaining formulation component compatibility.

The term “solvent vehicle” describes compositions that include both a volatile solvent system and non-volatile solvent system. The volatile solvent system is chosen so as to evaporate from the adhesive formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and/or various aliphatic resins and aromatic resins.

The terms “washable,” “washing” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol or combinations thereof. Surfactants can also be used in some embodiments.

An acceptable length of time with respect to “drying time” refers to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, the present invention is drawn to an adhesive formulation for dermal delivery of a drug can comprise a drug, a solvent vehicle, and a solidifying agent. The solvent vehicle can comprise a volatile solvent system including at least one volatile solvent and a non-volatile solvent system including at least two non-volatile solvents. The at least two non-volatile solvents of the non-volatile solvent system can facilitate transdermal delivery of the drug at a therapeutically effective rate over a sustained period of time, even after the non-volatile solvent system is substantially evaporated. The formulation can have viscosity suitable for application to the skin surface prior to evaporation of at least one volatile solvent, and can further be formulated such that when applied to the skin surface, the formulation forms a solidified layer after at least a portion of the volatile solvent system is evaporated. Sustained drug delivery from the solidified layer can also occur.

In an alternative embodiment, a method of dermally delivering a drug can comprise applying an adhesive solidifying formulation to a skin surface of a subject. The formulation can comprise a drug, solvent vehicle, and a solidifying agent. The solvent vehicle can comprise a volatile solvent system including at least one volatile solvent, and a non-volatile solvent system including at least two non-volatile solvents, wherein the non-volatile solvent system facilitates dermal delivery of the drug at a therapeutically effective rate over a sustained period of time. The formulation can have a viscosity suitable for application and adhesion to the skin surface prior to evaporation of the volatile solvent system Other steps include solidifying the formulation to form a solidified layer on the skin surface by at least partial evaporation of the volatile solvent system; and dermally delivering the drug from the solidified layer to the skin surface at therapeutically effective rates over a sustained period of time.

In another embodiment, a solidified layer for delivering a drug can comprise a drug, a non-volatile solvent system including at least two non-volatile solvents, wherein the non-volatile solvent system is capable of facilitating the delivery of the drug at therapeutically effective rates over a sustained period of time, and a solidifying agent. The solidified layer can be stretchable by 5% in at least one direction without cracking, breaking, or separating from a skin surface to which the layer is applied.

In further detail, the formulations can also contain substances that are solid or liquid at room temperatures, such as pH or ion-pairing agents. After evaporation of the volatile solvent system, most of the non-volatile solvent system should remain in the solidified layer for a period of time sufficient to adequately dermally deliver a given drug to, into, or through the skin of a subject at a sufficient flux for a period of time to provide a therapeutic effect. The non-volatile solvent system can also serve as a plasticizer of the solidified layer, so that the solidified layer is elastic and flexible.

Thus, these embodiments exemplify the present invention which is related to novel formulations, methods, and solidified layers that are typically in the initial form of semi-solids (including creams, gels, pastes, ointments, and other viscous liquids), which can be easily applied onto the skin as a layer, and can quickly (from 15 seconds to about 4 minutes under standard skin and ambient conditions) to moderately quickly (from about 4 to about 15 minutes under standard skin and ambient conditions) change into a solidified layer, e.g., a coherent and soft solid layer which can be peelable, for drug delivery. A solidified layer thus formed is capable of delivering drug to the skin, into the skin, across the skin, etc., at substantially constant rates, over an sustained period of time, e.g., hours to tens of hours, so that most of the active drug is delivered after the solidified layer is formed.

Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus capable of maintaining good contact with a skin surface, even if the skin is stretched during body movement, such as at a knee, finger, elbow, or other joints.

In selecting the various components that can be used, e.g., drug, solvent vehicle of volatile solvent system and non-volatile solvent system, solidifying agent(s), etc., various considerations can occur. For example, the volatile solvent system can be selected from pharmaceutically or cosmetically acceptable solvents known in the art. In one embodiment of the present invention, the volatile solvent system can include ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone or combinations thereof. In another embodiment of the present invention, the volatile solvent system can include iso-amyl acetate, denatured alcohol, methanol, propanol, isobutene, pentane, hexane, chlorobutanol, turpentine, cytopentasiloxane, cyclomethicone, methyl ethyl ketone, or combinations thereof. The volatile solvent system can include a mixture or combination of any of the volatile solvents set forth in the embodiments above.

Additionally, these volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %. In one aspect of the invention, the volatile solvent system comprises at least 10 wt % of the formulation. In another embodiment, the volatile solvent system comprises at least about 20 wt % of the formulation.

The volatile solvent system can also be chosen to be compatible with the non-volatile solvent system, solidifying agent, drug, and any other excipients that may be present. For example, polyvinyl alcohol (PVA) is not soluble in ethanol. Therefore, a volatile solvent which will dissolve PVA needs to be formulated in the solidified layer. For instance, water will dissolve PVA and can be utilized as a volatile solvent in a formulation; however, the drying time in such a formulation may be too long to certain applications. Therefore, a second volatile solvent (e.g., ethanol) can be formulated into the formulation to reduce the water content but maintain a sufficient amount of water to keep PVA in solution and thereby reduce the drying time for the formulation.

The non-volatile solvent system can also be chosen or formulated to be compatible with the solidifying agent, the drug, the volatile solvent, and any other ingredients that may be present. For example, the solidifying agent can be chosen so that it is dispersible or soluble in the non-volatile solvent system. Most non-volatile solvent systems and solvent vehicles as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a formulation containing PVA. With this combination, Span 20 can separate out of the formulation and form an oily layer on the surface of the solidified layer. Thus, appropriate solidifying agent/non-volatile solvent selections are desirable in developing a viable formulation and compatible combinations. It is not necessary that both the non-volatile solvents of the non-volatile solvent system be compatible with the solidifying agent. In some embodiments one of the non-volatile solvents of the non-volatile solvent system can be present to provide compatibility with the solidifying agent while a second non-volatile solvent can act as the flux enabling non-volatile solvent.

In further detail, the at least two non-volatile solvents that can be used to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In another embodiment the non-volatile solvent system can include benzoic acid, butyl alcohol, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers such as PEG-cetyl ether, PEG-stearyl ether, PEG-sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment, the non-volatile solvent system can include a combination or mixture of non-volatile solvents set forth in any of the above discussed embodiments.

In addition to these and other considerations, the non-volatile solvent system, or at least one of the non-volatile solvents in the non-volatile solvent system can also serve as plasticizer in the adhesive formulation so that when the solidified layer is formed, the layer is flexible, stretchable, and/or otherwise “skin friendly.”

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the volatile solvent is capable of irritating the skin, it would be helpful to use a non-volatile solvent that is capable of reducing skin irritation. Examples of solvents that are known to be capable of preventing or reducing skin irritation include, but are not limited to, glycerin, honey, and propylene glycol.

The feature of two non-volatile solvents in the non-volatile solvent system enhances the ability of the non-volatile solvent to provide therapeutically effective flux, while at the same time providing additional important characteristics which make the solidified formulations superior. As discussed in other areas of the application, non-volatile solvents can provide advantageous benefits such as acting as a plasticizer, improve adhesion, reducing skin irritation, inhibiting phase separation, and the like. In some embodiments it may be desirable to deliver two drugs which do not share a common flux-enabling non-volatile solvent. In such instances at least on of the at least two non-volatile solvents present in the non-volatile solvent system can act to promote the flux of one of the drugs while the other non-volatile solvent promotes the flux of the other drug. In such situations it may be desirable or necessary to include an additional non-volatile solvent which provides some of the other advantageous benefits discussed above.

The two or more non-volatile solvents of the non-volatile solvent system of the present invention may be such that the non-volatile solvents used independently are not flux-enabling non-volatile solvents for a drug but when formulated together become a flux-enabling non-volatile solvent. One possible reason for these initially non enabling non-volatile solvents to become enabling non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and propylene glycol/isostearic acid.

The selection of the solidifying agent can also be carried out in consideration of the other components present in the adhesive formulation. The solidifying agent can be selected or formulated to be compatible to the drug and the solvent vehicle (including the volatile solvent(s) and the non-volatile solvent system), as well as to provide desired physical properties to the solidified layer once it is formed. Depending on the drug, solvent vehicle, and/or other components that may be present, the solidifying agent can be selected from a variety of agents. In one embodiment, the solidifying agent can include polyvinyl alcohol with a MW range of 20,000-70,000 (Amresco), esters of polyvinylmethylether/maleic anhydride copolymer (ISP Gantrez ES-425 and Gantrez ES-225) with a MW range of 80,000-160,000, neutral copolymer of butyl methacrylate and methyl methacrylate (Degussa Plastoid B) with a MW range of 120,000-180,000, dimethylaminoethyl methacrylate-butyl methacrylate-methyl methacrylate copolymer (Degussa Eudragit E100) with a MW range of 100,000-200,000, ethyl acrylate-methyl methacrylate-trimethylammonioethyl methacrylate chloride copolymer with a MW greater than 5,000 or similar MW to Eudragit RLPO (Degussa), Zein (prolamine) with a MW greater than 5,000 such as Zein with a MW around 35,000 (Freeman industries), pregelatinized starch having a MW similar to Instant Pure-Cote B793 (Grain Processing Corporation), ethyl cellulose MW greater than 5,000 or MW similar to Aqualon EC N7, N10, N14, N22, N50, or N100 (Hercules), fish gelatin having a MW 20,000-250,000 (Norland Products), gelatin, other animal sources with MW greater than 5,000, acrylates/octylacrylamide copolymer MW greater than 5,000 or MW similar to National Starch, Chemical Dermacryl 79, or combinations thereof.

In another embodiment, the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, or combinations thereof. In a further embodiment, the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methacrylic acid-ethyl acrylate copolymers such as BASF's Kollicoat polymers, methacrylic acid and methacrylate based polymers such as poly(methacrylic acid), or combinations thereof. In another embodiment, the solidifying agent can include a combination of solidifying agents set forth in the any of the above discussed embodiments. Other polymers may also be suitable as the solidifying agent, depending on the solvent vehicle components, the drug, and the specific functional requirements of the given formulation. Other polymers may also be suitable as the solidifying agent, depending on the solvent vehicle components, the drug, and the specific functional requirements of the given formulation.

In one embodiment, the non-volatile solvent system and the solidifying agent(s) should be compatible with each other. Compatibility can be defined as i) the solidifying agent does not substantially negatively influence the function of the non-volatile solvent system, except for some reduction of flux; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent(s) can be from about 0.1:1 to about 10:1. In another aspect, the ratio between the non-volatile solvent system and the solidifying agent can be from about 0.5:1 to about 2:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface of the solidified layer. If the drug is very potent and the solidified layer has very high tensile strength, a layer as thin as 0.01 mm may be sufficient. If the drug has rather low potency and the solidified layer has low tensile strength, a layer as thick as 2-3 mm may be desirable. Thus, for most drugs and formulations, the appropriate thickness can be from about 0.01 mm to about 3 mm, but more typically, from about 0.05 mm to about 1 mm.

The flexibility and stretchability of a solidified layer can be desirable in some applications. In one aspect of the invention, the solidified layer is coherent, flexible, and continuous. Such flexible and coherent nature can greatly enhance the ease of use of the formulation. For instance, certain non-steroidal anti-inflammatory agents (NSAIDs) can be applied directly over joints and muscles for transdermal delivery into joints and muscles. However, skin areas over joints and certain muscle groups are often significantly stretched during body movements. Such movement prevents non-stretchable patches from maintaining good skin contact. Lotions, ointments, creams, gels, foams, pastes, or the like also may not be suitable for use for the reasons cited above. As such, in transdermal delivery of NSAIDs into joints and/or muscles, the solidifying formulations of the present invention can offer unique advantages and benefits. It should be pointed out that although good stretchability can be desirable in some applications. The solidifying formulations of the present invention do not always need to be stretchable, as certain applications of the present invention do not necessarily benefit from this property. For instance, if the formulation is applied on a small facial area overnight for treating acne, a subject would experience minimal discomfort and formulation-skin separation even if the solidified layer is not stretchable, as facial skin usually is not stretched very much during a sleep cycle.

A further feature of a formulation prepared in accordance with embodiments of the present invention is related to drying time. If a formulation dries too quickly, the user may not have sufficient time to spread the formulation into a thin layer on the skin surface before the formulation is solidified, leading to poor skin contact. If the formulation dries too slowly, the subject may have to wait a long time before resuming normal activities (e.g. putting clothing on) that may remove un-solidified formulation. Thus, it is desirable for the drying time to be longer than about 15 seconds but shorter than about 15 minutes, and preferably from about 0.5 minutes to about 5 minutes.

Other benefits of the solidified layers of the present invention include the presence of a physical barrier that can be formed by the material itself. For instance, local anesthetic agents and other agents such as clonidine may be delivered topically for treating pain related to neuropathy, such as diabetic neuropathic pain. Since many of such subjects feel tremendous pain, even when their skin area is only gently touched, the physical barrier of the solidified layer can prevent or minimize pain caused by accidental contact with objects or others.

These and other advantage can be summarized in the following non-limiting list of benefits, as follows. The solidified layers of the present invention can be prepared in an initial form that is easy to apply as a semisolid dosage form. Additionally, upon volatile solvent system evaporation, the resulting solidified layer is relatively thick and can contain much more active drug than a typical layer of traditional cream, gel, lotion, ointment, paste, etc., and further, is not as subject to unintentional removal. Further, as the solidified layer remains adhesive and optionally peelable, easy removal of the solidified layer can occur, usually without the aid of a solvent or surfactant. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched by 5%, or even 10% or greater, in at least one direction without cracking, breaking, and/or separating form a skin surface to which the solidified layer is applied. Still further, the solidified layer can be formulated to advantageously deliver drug and protect sensitive skin areas without cracking or breaking. Generally, the solidified layers made using the formulations of the present invention can be soft and coherent solids that are peelable from a skin surface as a single piece or as only a few large pieces relative to the application size. In other embodiments, the solidified layer can be removable by use of a solvent, such as water, alcohol, surfactant, or mixture thereof.

As a further note, it is a unique feature of the solidified layers of the present invention that they can keep a substantial amount of the non-volatile solvent system, which is optimized for delivering the drug, on the skin surface. This feature can provide unique advantages over existing products. For example, in some semi-solid formulations, upon application to a skin surface the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

Specific examples of applications that can benefit from the systems, formulations, and methods of the present invention are as follows. In one embodiment, a solidified layer including bupivacaine, lidocaine, or ropivacaine, can be formulated for treating diabetic and post herpetic neuralgia. Alternatively, dibucanine and an alpha-2 agonist such as clonidine can be formulated in a solidified layer for treating the same disease. In another embodiment, retinoic acid and benzoyl peroxide can be combined in a solidified layer for treating acne, or alternatively, 1 wt % clindamycin and 5 wt % benzoyl peroxide can be combined in a solidified layer for treating acne. In another embodiment, a retinol solidifying formulation (OTC) can be prepared for treating wrinkles, or a lidocaine solidifying formulation can be prepared for treating back pain. In another embodiment, a zinc oxide solidifying formulation (OTC) can be prepared for treating diaper rash, or an antihistamine solidified layer can be prepared for treating allergic rashes such as poison ivy.

Additional applications include delivering drugs for treating certain skin conditions, e.g., dermatitis, psoriasis, eczema, skin cancer, viral infections such as cold sore, genital herpes, shingles, etc., particularly those that occur over joints or muscles where a transdermal patch may not be practical. For example, solidifying formulations containing imiquimod can be formulated for treating skin cancer, common and genital warts, and actinic keratosis. Solidifying formulations containing antiviral drugs such as acyclovir, penciclovir, famciclovir, valacyclovir, steroids, behenyl alcohol can be formulated for treating herpes viral infections such as cold sores on the face and genital areas. Solidifying formulations containing non-steroidal anti-inflammatory drugs (NSAIDs), capsaicin, alpha-2 agonists, and/or nerve growth factors can be formulated for treating soft tissue injury and muscle-skeletal pains such as joint and back pain of various causes. As discussed above, patches over these skin areas typically do not have good contact over sustained period of time, especially for a physically active subject, and may cause discomfort. Likewise, traditional semi-solid formulations such as creams, lotions, ointments, etc., may prematurely stop the delivery of a drug due to the evaporation of solvent and/or unintentional removal of the formulation. The solidified adhesive formulations of the present invention address the shortcomings of both of these types of delivery systems.

A further embodiment involves a formulation containing at least one alpha-2 agonist drug, at least one tricyclic antidepressant agent, and/or at least one local anesthetic drug which is applied topically to treat neuropathic pain. The drugs are gradually released from the formulation to provide pain relief over a sustained period of time. The formulation can become a coherent, soft solid after 2-5 minutes and remains adhered to the skin surface for the length of its application. It is easily removed any time after drying without leaving residual formulation on the skin surface.

Another embodiment involves a formulation containing capsaicin which is applied topically to treat neuropathic pain. The capsaicin is gradually released from the formulation for treating this pain over a sustained period of time. The formulation can become a coherent, soft solid after 2-5 minutes and remains adhered to the skin surface for the length of its application. It is easily removed any time after drying without leaving residual formulation on the skin surface.

Another embodiment involves solidifying formulations containing tazorac for treating stretch marks, wrinkles, sebaceous hyperplasia, seborrheic keratosis. In another embodiment, solidifying formulations containing glycerol can be made so as to provide a protective barrier for fissuring on finger tips.

Still another embodiment can include a formulation containing a drug selected from the local anesthetic class such lidocaine and ropivacaine or the like, or NSAID class, such as ketoprofen, piroxicam, diclofenac, indomethacin, or the like, which is applied topically to treat symptoms of back pain, muscle tension, or myofascial pain or a combination thereof. The local anesthetic and/or NSAID is gradually released from the formulation to provide pain relief over a sustained period of time. The formulation can become a coherent, soft solid after about 2-5 minutes and remains adhered to the skin surface for the length of its application. It is easily removed any time after drying without leaving residual formulation on the skin surface.

A similar embodiment can include a formulation containing drugs capsaicin and a local anesthetic drug which is applied topically to the skin to provide pain relief. Another embodiment can include a formulation containing the combination of a local anesthetic and a NSAID. In both of the above embodiments the drugs are gradually released from the formulation to provide pain relief over a sustained period of time. The formulation can become a coherent, soft solid after 2-4 minutes and remains adhered to the skin surface for the length of its application. It is easily removed any time after drying without leaving residual formulation on the skin surface.

In another embodiment, solidifying formulations for the delivery of drugs that treat the causes or symptoms of diseases involving joints and muscles can also benefit from the systems, formulations, and methods of the present invention. Such diseases that may be applicable include, but not limited to, osteoarthritis (OA), rheumatoid arthritis (RA), joint and skeletal pain of various other causes, myofascial pain, muscular pain, and sports injuries. Drugs or drug classes that can be used for such applications include, but are not limited to, non-steroidal anti-inflammatory drugs (NSAIDs) such as ketoprofen and diclofanec, COX-2 selective NSAIDs and agents, COX-3 selective NSAIDs and agents, local anesthetics such as lidocaine, bupivacaine, ropivacaine, and tetracaine, steroids such as dexamethasone.

Delivering drugs for the treatment of acne and other skin conditions can also benefit from principles of the present invention, especially when delivering drugs having low skin permeability. Currently, topical retinoids, peroxides, and antibiotics for treating acne are mostly applied as traditional semisolid gels or creams. However, due to the shortcomings as described above, sustained delivery over many hours is unlikely. For example, clindamycin, benzoyl peroxide, and erythromycin may be efficacious only if sufficient quantities are delivered into hair follicles. However, a traditional semisolid formulation, such as the popular acne medicine benzaclin gel, typically loses most of its solvent (water in the case of benzaclin) within a few minutes after the application. This short period of a few minutes likely substantially compromises the sustained delivery of the drug. The formulations of the present invention typically do not have this limitation.

In another embodiment, the delivery of drugs for treating neuropathic pain can also benefit from the methods, systems, and formulations of the present invention. A patch containing a local anesthetic agent, such as Lidoderm™, is widely used for treating neuropathic pain, such as pain caused by post-herpetic neuralgia and diabetes induced neuropathic pain. Due to the limitations of the patch as discussed above, the solidified layers prepared in accordance with the present invention provide some unique benefits, as well as provide a potentially less expensive alternative to the use of a patch. Possible drugs delivered for such applications include, but are not limited to, local anesthetics such as lidocaine, prilocaine, tetracaine, bupivicaine, etidocaine; and other drugs including capsaicin and alpha-2 agonists such as clonidine, dissociative anesthetics such as ketamine, tricyclic antidepressants such as amitriptyline.

As set forth in part above, the solidifying formulations of the present invention can be formulated to treat a variety of conditions and disease such as musculoskeletal pain, neuropathic pain, alopecia, skin disease including dermatitis and psoriasis as well as skin restoration (cosmetic skin treatment), and infections including viral, bacterial, and fungal infection. As such, the formulations can deliver a wide ranging number and types of drugs and active agents.

In one embodiment, the solidifying formulation can be formulated to include acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, Isotretinoin, clobetasol propionate, halobetasol propionate, ketoprofen, piroxicam, diclofenac, indomethacin, imiquimod, salicylic acid, benzoic acid, or combinations thereof

In another embodiment, the formulation can include an antifungal drug such as amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, and undecylenate, or combinations thereof.

In another embodiment, the formulation can include an antifungal drug such as acyclovir, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, or combinations thereof.

When the formulation is intended to provide antibacterial treatment it can be formulated to include an antibacterial drug such as erythromycin, clindamycin, tetracycline, bacitracin, neomycin, mupirocin, polymyxin B, quinolones such as ciproflaxin, or combinations thereof.

When the formulation is intended to relieve pain, particularly neuropathic pain, the formulation can include a local anesthetic such as lidocaine, bupivacaine, ropivacaine, and tetracaine; an alpha-2 agonists such as clonidine. When the formulation is intended to treat pain associated with inflammation it can be formulated to include an non-steroidal anti-inflammatory drug such as ketoprofen, piroxicam, diclofenac, indomethacin, COX inhibitors general COX inhibitors, COX-2 selective inhibitors, COX-3 selective inhibitors, or combinations thereof

In another embodiment, the formulation can be formulated to treat skin disorders or blemishes by including active agents such as anti-acne drugs such as clindamycin and benzoyl peroxide, retinol, vitamin A derivatives such as tazarotene and isotretinoin, cyclosporin, anthralin, vitamin D3, cholecalciferol, calcitriol, calcipotriol, tacalcitol, calcipotriene, or combinations thereof.

In yet another embodiment, the delivery of medication for treating warts and other skin conditions would also benefit from long periods of sustained drug delivery. Examples of anti-wart compounds include but are not limited to:imiquimod, rosiquimod, keratolytic agents: salicylic acid, alpha hydroxy acids, sulfur, rescorcinol, urea, benzoyl peroxide, allantoin, tretinoin, trichloroacetic acid, lactic acid, benzoic acid, or combinations thereof.

A further embodiment involves the use of the solidifying formulations for the delivery of sex steroids including but not limited to progestagens consisting of progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, androgens consisting of testosterone, methyl testosterone, oxandrolone, androstenedione, dihydrotestosterone, estrogens such as estradiol, ethniyl estradiol, estiol, estrone, conjugated estrogens, esterified estrogens, estropipate, or combinations thereof.

Non-sex steroids can also be delivered using the formulations of the present invention. Examples of such steroids include but are not limited to betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, or combinations thereof.

A further embodiment involves controlled delivery of nicotine for treating nicotine dependence among smokers and persons addicted to nicotine. Formulations of the present invention would be a cost effective way of delivering therapeutic amounts of nicotine transdermally.

Another embodiment involves using the formulation to deliver anti-histamine agents such as diphenhydramine and tripelennamine. These agents would reduce itching by blocking the histamine that causes the itch and also provide relief by providing topical analgesia.

Other drugs which can be delivered using the solidifying formulations of the present invention include but are not limited to tricyclic anti-depressants such as amitriptyline; anticonvulsants such as carbamazepine and alprazolam; N-methyl-D-aspartate (NMDA) antagonists such as ketamine; 5-HT2A receptor antagonists such as ketanserin; and immune modulators such as tacrolimus and picrolimus. Other drugs that can be delivered using the formulations and methods of the current invention include humectants, emollients, and other skin care compounds.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Example 2

Human cadaver skin is used as a membrane to select a non-volatile solvent for clobetasol propionate. In vitro methodology is described in Example 1. About 200 mcL of 0.1% (w/w) solution of clobetasol in various non-volatile solvents is added to the donor compartment of Franz cells. Results obtained after LC analysis are shown in Table 4.

TABLE 4
Non volatile solvents for clobetasol propionate

| | Skin Flux* |
|---|---|
| Non-volatile solvent system | (ng/cm²/h) |
| Propylene Glycol | 3.8 ± 0.4 |
| Glycerol | 7.0 ± 4.1 |
| Light Mineral Oil | 31.2 ± 3.4 |
| Isostearic Acid (ISA) | 19.4 ± 3.2 |
| Ethyl Oleate | 19.4 ± 1.6 |
| Olive Oil | 13.6 ± 3.3 |
| Propylene Glycol/ISA (9:1) | 764.7 ± 193.9 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

All the neat non-volatile solutions studied have an average flux of less than 40 ng/cm²/hr over the 30 hour time period. Propylene glycol and glycerol have the lowest permeation for clobetasol propionate. A mixture of propylene glycol and isostearic acid at a weight ratio of 9:1 have significantly higher flux than either of the solvents alone or with the other solvents tested. The average flux is 20 times higher than that with light mineral oil which is the best non-mixed solvent. Hence, for clobetasol propionate, propylene glycol/isostearic acid combination is a good candidate for a non-volatile solvent system.

### Examples 3-8

Adhesive formulations containing 0.05% (w/w) clobetasol propionate with propylene glycol and isostearic acid as non volatile solutions and various solidifying agents are prepared. The formulations are prepared from the ingredients as shown in Table 5.

TABLE 5
Solidifying formulation components

| | | | | Percent | Percent | |
|---|---|---|---|---|---|---|
| Ex- | | Percent | Percent | Propylene | Isostearic | Percent |
| ample | Polymer | Polymer | Ethanol | Glycol | Acid | Water |
| 3 | Polyvinyl | 20 | 30 | 19.6 | 0.4 | 30 |
| | Alcohol | | | | | |
| 4 | Shellac | 50 | 30 | 19.6 | 0.4 | 0 |
| 5 | Dermacryl | 65.76 | 21.16 | 12.76 | 0.26 | 0 |
| | 79 | | | | | |
| 6 | Eudragit | 50 | 30 | 19.6 | 0.40 | 0 |
| | E100 | | | | | |
| 7 | Eudragit | 50 | 30 | 19.6 | 0.40 | 0 |
| | RLPO | | | | | |
| 8 | Gantrez | 14.3 | 57.1 | 28 | 0.6 | 0 |
| | S97 | | | | | |

Each of the compositions shown above are studied for flux of clobetasol propionate as shown in Table 6 as follows:

TABLE 6
Steady state flux of Clobetasol propionate
through human cadaver skin at 35° C.

| | Skin Flux* |
|---|---|
| Formulation | (ng/cm²/h) |
| Example 3 | 87.8 ± 21.4 |
| Example 4 | 9.7 ± 2.4 |
| Example 5 | 8.9 ± 0.8 |
| Example 6 | 3.2 ± 1.7 |
| Example 7 | 20.2 ± 18.6 |
| Example 8 | 147.5 ± 38.8 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

As seen from Table 6 formulation described in Example 3 that contains polyvinyl alcohol as solidifying agent has high flux of clobetasol propionate. Polyvinyl alcohol is known to form stretchable films and it is likely that this formulation will have acceptable wear properties. The toughness of the resulting film can be modified by adding appropriate plasticizers if needed. Tackiness can also be modified by adding appropriate amounts of tackifier or by adding appropriate amounts of another solidifying agent such as Dermacryl 79.

Regarding formulation described in Example 8, a higher percentage of ethanol is needed to dissolve the polymer. However, the solidifying agent used in Example 8 provides the highest flux of clobetasol propionate among the solidifying agents studied. The wear properties of this formulation can be modified by adding appropriate levels of other ingredients including but not limited to plasticizers, tackifiers, non-volatile solvents and or solidifying agents.

### Example 9

Formulations of acyclovir in various non-volatile solvent systems are evaluated. Excess acyclovir is present. The permeation of acyclovir from the test formulations through HMS is presented in Table 7 below.

TABLE 7

| | Skin Flux* |
|---|---|
| Non-volatile solvent system | (mcg/cm²/h) |
| Isostearic acid | 0.1 ± 0.09 |
| Isostearic acid + 10% trolamine | 2.7 ± 0.6 |
| Isostearic acid + 30% trolamine | 7 ± 2 |
| Olive oil | 0.3 ± 0.2 |
| Olive oil + 11% trolamine | 3 ± 3 |
| Olive oil + 30% trolamine | 0.3 ± 0.2 |
| Oleic acid | 0.4 ± 0.3 |
| Oleic acid + 10% trolamine | 3.7 ± 0.5 |
| Oleic acid + 30% trolamine | 14 ± 5 |
| Ethyl oleate | 0.2 ± 0.2 |
| Ethyl oleate + 10% trolamine | 0.2 ± 0.2 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of acyclovir from the above non-volatile solvents are obtained by placing 200 mcL on the stratum corneum side (donor) of hairless mouse skin. The in vitro studies are carried out as described in Example 1. The surprising result showed the polyethylene glycol 400, span 80, ethyl oleate, or ethyl oleate plus trolamine are not flux-enabling solvents for acyclovir (e.g., steady state flux values significantly less than the steady state flux of acyclovir in the marketed product noted in Table 2, where the flux was about 3 mcg/cm²/h). However, the combination of isostearic acid and trolamine or oleic acid and increasing amounts of trolamine are flux-enabling solvents for acyclovir. As can be seen, the highest flux was achieved using 30% trolamine with oleic acid as the non-volatile solvent system.

### Examples 10-13

Prototype solidifying formulations are prepared as follows. Several acyclovir solidifying formulations are prepared in accordance with embodiments of the present invention in accordance with Table 8, as follows:

TABLE 8

Example

| | | | |
|---|---|---|---|
| 10 | 11 | 12 | 13 |

% by weight

| | | | | |
|---|---|---|---|---|
| Ethanol | 21 | 25 | 28 | 29.5 |
| Eudragit RL-PO | 15 | 18 | 20 | 21.0 |
| Isostearic Acid | 31 | 36 | 39 | 42.0 |
| Trolamine | 30 | 18 | 10 | 4.7 |
| Acyclovir | 3 | 3 | 3 | 2.8 |

In Examples 10-13, the compositions in Table 6 are prepared as follows. Eudragit RL-PO and ethanol are combined in a glass jar and heated with stirring until the RL-PO is dissolved. The isostearic acid and trolamine is added to the RL-PO/ethanol mixture and the mixture is vigorously stirred. Once a uniform mixture is obtained, acyclovir is added to the mixture and the formulation is vigorously mixed.

### Examples 14-15

Prototype peel formulations are prepared as follows. Several acyclovir solidifying formulations are prepared in accordance with embodiments of the present invention in accordance with Table 9, as follows:

TABLE 9

Example

| | |
|---|---|
| 14 | 15 |

% by weight

| | | |
|---|---|---|
| Ethanol | 26 | 21 |
| Eudragit RL-PO | 44 | 15 |
| Isostearic Acid | 26 | 31 |
| Diisopropanol Amine | 2 | — |
| Neutral TE Polyol | — | 30 |
| Acyclovir | 2 | 3 |

The compositions of Examples 14 and 15 as shown in Table 8 are prepared as follows. Eudragit RL-PO and ethanol are combined in a glass jar and heated with stirring until the RL-PO is dissolved. The isostearic acid and diisopropanol amine or Neutrol TE Polyol (BASF) is added to the RL-PO/ethanol mixture and the mixture is vigorously stirred. Once a uniform mixture is obtained, acyclovir is added to the mixture and the formulation is vigorously mixed.

### Examples 16-17

Prototype solidifying formulations are prepared as follows. Several acyclovir solidifying formulations are prepared in accordance with embodiments of the present invention in accordance with Table 10, as follows:

TABLE 10

Example

| | |
|---|---|
| 16 | 17 |

% by weight

| | | |
|---|---|---|
| Ethanol | 59.6 | 58 |
| EC-N7 | 19.9 | — |
| EC-N100 | — | 19 |
| Trolamine | 7.6 | 9 |
| Isostearic Acid | 7.7 | 9 |
| Acyclovir | 5.2 | 5 |

In Examples 16-17 the compositions in Table 10 are prepared as follows. Ethyl cellulose ECN7 or ethyl cellulose ECN100 and ethanol are combined in a glass jar and heated with stirring until the solid cellulose is dissolved. The isostearic acid and trolamine is added to the cellulose/ethanol mixture and the mixture is vigorously stirred. Once a uniform mixture is obtained, acyclovir is added to the mixture and the formulation is vigorously mixed.

### Example 18

The formulations of Examples 10-17 are tested in a hairless mouse skin (HMS) in vitro model described in Example 1. Table 11 shows data obtained using the experimental process outlined above.

TABLE 11
Steady-state flux (J) of Acyclovir through HMS

| | J* | Ratio to |
|---|---|---|
| Formulation | (μg/cm²/h) | Control |
| Example 10 | 12 ± 5 | 6 |
| Example 11 | 19 ± 1 | 8 |
| Example 12 | 8 ± 1 | 4 |
| Example 13 | 1 ± 1 | 0.5 |
| Example 14 | 0.7 ± 0.3 | 0.35 |
| Example 15 | 1 ± 0.9 | 0.5 |
| Example 16 | 2 ± 1 | 1 |
| Example 17 | 19 ± 7 | 8 |
| Zovirax Cream | 2 ± 0.4 | 1 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

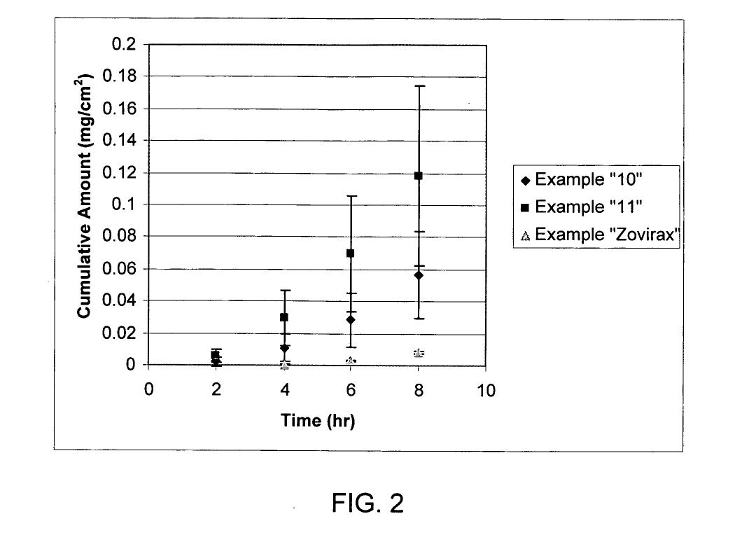

The formulations of the invention shown above generally provide for significant penetration of the active ingredient, and further, the formulations of Examples 10-12 and 17 are found to be much greater in permeability than the marketed product Zovirax Cream. The quantity of acyclovir that permeated across the HMS stratum corneum over time for Examples 10, 11, and Zovirax Cream are shown in FIG. 2. Each value shown indicates the mean±SD of at least three experiments.

Examples 10-13 show the impact of the trolamine to isostearic acid (ISA) ratio on acyclovir flux enhancement. The optimal ISA:trolamine ratio is 1:1 to 2:1 and ratio greater than 4:1 show a significant decrease in the acyclovir skin flux. Additions of diisopropanol amine and Neutrol in place of trolamine (Examples 14 and 15 in the formulation show a significant decrease in acyclovir flux values. This may be due to a specific chemical interaction between trolamine and ISA creating an environment within the formulation which facilitates higher skin flux. Examples 16 and 17 utilize a different solidifying agent to evaluate the impact of the solidifying agent on acyclovir flux. Surprisingly, Example 16 shows a significant decrease in acyclovir skin flux, but Example 17, which differed from Example 16 only by the molecular weight of the solidifying agent, shows no impact on acyclovir skin flux compared to a similar ISA:trolamine ratio in Example 10.

As can be seen from FIG. 2, Examples 10 and 11 show sustained delivery of acyclovir up to 8 hours, it is reasonable to assume based on the drug load and the continued presence of the non volatile solvent that the delivery of acyclovir would continue at the reported flux values for as long as the subject desires to leave the solidifying formulation affixed to the skin.

### Examples 19-21

Prototype solidifying formulations are prepared as follows. Several solidifying formulations are prepared in accordance with embodiments of the present invention in accordance with Table 12, as follows:

TABLE 12

| Example | | |
|---|---|---|
| 19 | 20 | 21 |

% by weight
Volatile Solvents

| | | | |
|---|---|---|---|
| Ethanol | 25 | 24 | 43 |
| Water | | | 22 |

Solidifying agents

| | | | |
|---|---|---|---|
| Eudragit RL-PO | 18 | 40 | |
| Polyvinyl Alcohol | | | 14 |

Non-volatile solvents

| | | | |
|---|---|---|---|
| Glycerol | | 12 | 14 |
| Propylene Glycol | | 4 | |
| Polyethylene glycol | | | 6 |
| Isostearic Acid | 36 | 13 | |
| Trolamine | 18 | 4 | |

Drug

| | | | |
|---|---|---|---|
| Acyclovir | 3 | | |
| Ropivacaine | | 3 | |
| Testosterone | | | 1 |

Solidifying formulations of Examples 19-21 are prepared in the following manner:
The solidifying agents are dissolved in the volatile solvent (e.g., dissolve polyvinyl alcohol in water, Eudragit polymers in ethanol),
The non-volatile solvent is mixed with the solidifying agent/volatile solvent mixture.
The resulting solution is vigorously mixed well for several minutes.
The drug is then added and the solidifying formulation is mixed again for several minutes.

In all the Examples noted above, the flux-enabling non-volatile solvent/solidifying agent/volatile solvent combination is compatible as evidenced by a homogeneous, single phase system that exhibited appropriate drying time, and provided a stretchable solidified layer and steady state flux for the drug (see Example 22 below).

### Example 22

The formulations of the examples are tested in a hairless mouse skin (HMS) or HEM in vitro model described in Example 1. Table 13 shows data obtained using the experimental process outlined above.

TABLE 13
Steady-state flux (J)

| | J* |
|---|---|
| Formulation | (μg/cm²/h) |
| Example 19 | 19 ± 1*** |
| Example 20 | 32 ± 2*** |
| Example 21 | 4 ± 1*** |

*Skin flux measurements represent the mean and standard deviation of three determinations.
**Data gathered using human epidermal membrane.
***Flux measurements reported were determined from the linear region of the cumulative amount versus time plots. The linear region was observed to be between 4-8 hours. If experimental conditions allowed, the steady-state delivery would likely continue well beyond 8 hours.
****Flux measurements reported were determined from the linear region of the cumulative amount versus time plots. The linear region was observed to be between 6-28 hours. If the experiment was continued it is anticipated the steady state would continue.

Acyclovir, ropivacaine, and testosterone have surprisingly higher steady state flux values when the flux-enabling non-volatile solvent is incorporated into the solidifying formulation. It is speculated that the higher flux values may be the result of contributions of the volatile solvent or the solidifying agent impacting the chemical environment (e.g., increasing solubility) of the drug in the solidifying formulation resulting in higher flux values.

### Example 23

A formulation with the following composition: 10.4% polyvinyl alcohol, 10.4% polyethylene glycol 400, 10.4% polyvinyl pyrrolidone K-90, 10.4% glycerol, 27.1% water, and 31.3% ethanol was applied onto a human skin surface at an elbow joint and a finger joint, resulting in a thin, transparent, flexible, and stretchable solidified layer. After a few minutes of evaporation of the volatile solvents (ethanol and water), a solidified layer that was peelable was formed. The non-volatile solvent system of polyethylene glycol and glycerol acts a plasticizer in the formulation. The stretchable solidified layer had good adhesion to the skin and did not separate from the skin on joints when bent, and could easily be peeled away from the skin.

### Examples 24-26

Three formulations similar to the formulation in Example 27 (replacing ropivacaine base with ropivacaine HCl) are applied on the stratum corneum side of freshly separated hairless mouse skin. The in vitro flux is determined for each formulation as outlined in Example 1. The formulation compositions are noted in Table 14 below.

TABLE 14

| Example | | |
|---|---|---|
| 24 | 25 | 26 |

% by weight

| | | | |
|---|---|---|---|
| PVA | 15 | 15 | 15 |
| Water | 23 | 23 | 23 |
| Ethylcellulose N-100 | 11 | 11 | 11 |
| Ethanol | 33 | 33 | 33 |
| Span 20 | 11 | | |
| Polyethylene Glycol 400 | | 11 | |
| Tween 40 | | | 11 |
| Tromethamine | 4 | 4 | 4 |
| Ropivacaine HCl | 3 | 3 | 3 |
| Avg. Flux* (mcg/cm2/h) | 15 ± 1 | 4.7 ± 0.3 | 3.4 ± 0.7 |

*Flux values represent the mean and standard deviation of three determinations. Flux measurements reported were determined from the linear region of the cumulative amount versus time plots. The linear region was observed to be between 4-9 hours. If the experiment was continued it is anticipated the steady state would continue.

Since all three formulations have the exact same compositions of solidifying agent, volatile solvents, and flux-enabling non-volatile solvent. The only difference is which flux-enabling non-volatile solvent is used it is reasonable to conclude that for ropivacaine HCl that Span 20, polyethylene glycol 400, and Tween 40 qualify as flux-enabling non-volatile solvents.

Addition of tromethamine and Span 20 in example 30 produced a flexible coherent solid that was much less brittle than a formulation containing no non-volatile solvents.

### Examples 27-31

A solidifying formulation for dermal delivery of imiquimod is prepared which includes a specified amount of imiquimod in an excipient mixture to form an adhesive formulation in accordance with embodiments of the present invention. The solidifying formulations contained the following components:

TABLE 15
Imiquimod peelable formulation ingredients

| | Example | | | | |
|---|---|---|---|---|---|
| Ingredients* | 27 | 28 | 29 | 30 | 31 |
| PVA | 12 | 21.5 | | | |
| Plastoid B** | | | 22.7 | 21.1 | 21 |
| Pemulen TR-2 | 0.3 | 0.3 | | | |
| Water | 62.7 | 34.4 | 2.8 | | |
| Isopropanol | | | 42.5 | 42.5 | 41.7 |
| ISA (Isostearic | 19 | 35.2 | 9.2 | 28.2 | 27.8 |
| Acid) | | | | | |
| Span 20 | | | 8.5 | | |
| Trolamine | 2 | 3.6 | 6.1 | | |
| Triacetin | | | 4.2 | 4.2 | 4.2 |
| Imiquimod | 4 | 5 | 4 | 4 | 5.3 |

*Ingredients are noted as weight percent.
**Polymer from Degussa

These formulations are applied to HMS skin as described in Example 1, and the imiquimod flux is measured. A summary of the results from in vitro flux studies carried out with the formulations in Examples 27-31 are listed in Table 16.

TABLE 16
Steady-state flux of imiquimod through hairless mouse skin
from various adhesive formulations at 35° C.

| | Average flux | Ratio to |
|---|---|---|
| Formulation | mcg/cm²/h* | Control** |
| Example 27 | 0.7 ± 0.09 | 0.7 |
| Example 28 | 0.52 ± 0.06 | 0.6 |
| Example 29 | 0.40 ± 0.08 | 0.4 |
| Example 30 | 0.5 ± 0.1 | 0.5 |
| Example 31 | 0.8 ± 0.1 | 0.9 |
| Aldara (control) | 0.92 ± 0.02 | |

*The flux values represent the mean and SD of three determinations
**Ratio to control calculated by dividing the flux value for each Example by the flux value for Aldara control flux.

Regarding the formulation described in Examples 27 and 28, water is used as the volatile solvent, and the ISA, trolamine mixture is used as the non-volatile solvent system. Through experimentation, it is determined that ISA and Span 20 provide the appropriate solubility for the drug, however, these non-volatile solvents are hydrophobic and not compatible with the volatile solvent system used to dissolve the solidifying agent PVA. An emulsifier Pemulen TR-2 was used to emulsify the non-volatile solvents into the water phase. Further, in this embodiment, ISA and trolamine act as a plasticizer in the peelable formulation after the water (volatile solvent) has evaporated. The steady state flux of formulation Examples 27 and 28 demonstrate the importance of the amount of non-volatile solvent in added to the formulation in dictating the flux-generating power of the entire formulation. Formulation Examples 29-31 utilize a different solidifying agent which is compatible in a non-aqueous volatile solvent system (isopropanol). The selection of non-volatile solvent system ISA/triacetin or ISA/Span 20/trolamine/triacetin combination showed no change in the in vitro flux. The increase in vitro flux is shown to be influenced by an increase in the amount of imiquimod present in the formulation. At imiquimod levels above 4% the drug is saturated in the solidifying formulation. The increase in vitro flux as a function of increased drug addition (Examples 30 and 31) may be due to the increased solubility of drug in the solidified formulation once the volatile solvent is evaporated off.

Example 29 demonstrated comparable imiquimod flux to the other formulation Examples, but the importance of the non-volatile solvent system and solidifying agent compatibility necessitated the removal of trolamine because this non-volatile solvent negatively influenced the function of the Plastoid B polymer.

### Example 32-35

A solidifying formulation for dermal delivery of imiquimod is prepared which includes a specified amount of imiquimod in an excipient mixture to form an adhesive formulation in accordance with embodiments of the present invention. The solidifying formulations contained the following components:

TABLE 17
Imiquimod formulation ingredients

Example

| Ingredients* | 32 | 33 | 34 | 35 |
|---|---|---|---|---|
| PVA | 10.1 | | | |
| Plastoid B** | | 17.5 | | |
| Eudragit RL | | | 16.2 | 24.8 |
| PO | | | | |
| Pemulen | 0.3 | | | |
| TR-2 | | | | |
| Water | 52.9 | | | |
| Isopropanol | | 35.1 | | |
| Ethanol | | | 32.4 | 38.6 |
| ISA | 16.8 | 23.4 | 23.1 | 27.6 |
| (Isostearic | | | | |
| Acid) | | | | |
| Salicylic | 15.2 | 16.4 | 16.2 | |
| Acid | | | | |
| Trolamine | 1.7 | | | |
| Triacetin | | 3.5 | 3.5 | 4.1 |
| Imiquimod | 3.0 | 4.1 | 4.0 | 4.8 |

*Ingredients are noted as weight percent.
**Polymer from Degussa

These formulations are applied to HMS skin as described in Example 1, and the imiquimod flux is measured. A summary of the results from in vitro flux studies carried out with the formulations in Examples 32-35 are listed in Table 18.

TABLE 18
Steady-state flux of Imiquimod through hairless mouse skin
from various adhesive formulations at 35° C.

| | Average flux | Ratio to |
|---|---|---|
| Formulation | mcg/cm²/h* | Control** |
| Example 32 | 1 ± 1 | 1.1 |
| Example 33 | 4.5 ± 0.4 | 5 |
| Example 34 | 3.8 ± 0.5 | 4.2 |
| Example 35 | 0.8 ± 0.2 | 0.9 |
| Aldara | 0.9 ± 0.02 | 1 |

*The flux values represent the mean and SD of three determinations
**Ratio to control calculated by dividing the flux value for each Example by the flux value for Aldara control flux.

In vitro flux of Examples 32-35 is substantially increased compared to the Aldara control. The reason for the improved in vitro flux values is attributed to the addition of salicylic acid. Improved in vitro flux of imiquimod in Examples 32-35 is thought to be due to an ion pair interaction between imiquimod and salicylic acid. The ion pair mechanism is thought that the lipophilicity of the counter ion (salicylic acid) improves the flux of imiquimod across the stratum corneum because it makes imiquimod less ‘comfortable’ in the formulation. Comparison of the flux of Examples 32-34 show that the selection of the polymer and/or volatile solvents will impact the flux of imiquimod. Example 32 contains PVA and water, one or both of these elements may contribute to an unfavorable medium in which the ion pair can form resulting in a negligible increase in imiquimod flux versus the Aldara control.

### Example 36

To demonstrate the ability of the solidified formulations to reduce the transepidermal water loss (TEWL) the following experiment was conducted.

A placebo PVA formulation was applied to the top of the hand and the TEWL was measured on a site immediately adjacent to the solidified layer and on top of the solidified layer. The TEWL measurement of the site covered by the solidified layer was 33% lower than the untreated skin site.

Placebo Plastoid B formulation similar to the formulation described in Example 5 was applied to the top of the hand and the TEWL was measured on a side immediately adjacent to the solidified layer and on top of the solidified layer. The TEWL measurement on the site covered by the solidified layer was 30% lower than the untreated skin site.

### Examples 37-38

A solidifying formulation for dermal delivery of ropivacaine is prepared which includes a specified amount of ropivacaine in an excipient mixture to form an adhesive formulation in accordance with embodiments of the present invention. The solidifying formulations contained the following components:

TABLE 19
Ropivacaine formulation ingredients.

Examples

| Ingredients* | 37 | 38 |
|---|---|---|
| Eudragit RL-100 | 39.6% | 39.6% |
| Ethanol | 23.7% | 23.6% |
| ISA (Isostearic Acid) | 13.5% | 13.5% |
| PG (Propylene Glycol) | 7.9% | 4.0% |
| Trolamine | 4.0% | 4.0% |
| Glycerol | 7.9% | 11.9% |
| Ropivacaine | 3.4% | 3.4% |

*Ingredients are noted as weight percent.

These formulations are applied to HMS skin as described in Example 1, and the ropivacaine flux is measured. A summary of the results from in vitro flux studies carried out with the formulations in Examples 37 and 38 is listed in Table 20.

TABLE 20
Steady-state flux of Ropivacaine through hairless mouse skin
from various adhesive formulations at 35° C.

| | Average flux |
|---|---|
| Formulation | mcg/cm²/h* |
| Example 37 | 36 ± 5 |
| Example 38 | 32 ± 2 |

*The flux values represent the mean and SD of three determinations

Regarding the formulation described in Examples 37 and 38, ethanol is used as the volatile solvent, and the ISA, glycerol, trolamine, and PG mixture is used as the non-volatile solvent system. Through experimentation, it is determined that ISA and propylene glycol used together to provide the appropriate solubility for the drug, while being compatible with the Eudragit RL-100 solidifying agent. Further, in this embodiment, ISA, PG and glycerol serve as a plasticizer in the peelable formulation after the ethanol (volatile solvent) has evaporated. The steady state flux of ropivacaine from formulation Examples 37 and 38 demonstrate the importance of the non-volatile solvent in dictating the flux-generating power of the entire formulation.

### Example 39

A formulation for dermal delivery of lidocaine is prepared which includes a saturated amount of lidocaine in an excipient mixture to form an adhesive formulation in accordance with embodiments of the present invention. The solidifying formulation is prepared from the ingredients as shown in Table 26.

TABLE 21
Lidocaine formulation components

| | Example |
|---|---|
| Ingredients* | 39 |
| PVA | 11.7 |
| Eudgragit E-100** | 11.7 |
| PVP-K90 | 5.8 |
| Glycerol | 8.8 |
| PEG-400 | 8.8 |
| Water | 23.8 |
| Ethanol | 23.8 |
| Lidocaine | 5.6 |

*Ingredients are noted as weight percent.
**from Rohm & Haas.

TABLE 22
Steady-state flux of Lidocaine through hairless mouse skin
from various adhesive formulations at 35° C.

| | Average flux |
|---|---|
| Formulation | mcg/cm²/h* |
| Example 39 | 47 ± 3 |

The adhesive formulation of lidocaine formulation in the present example has similar physical properties to the formulations in examples noted above. The transdermal flux across hairless mouse skin is acceptable and steady-state delivery is maintained over 8 hours.

### Examples 40-43

A formulation for dermal delivery of amitriptyline and a combination of amitripyline and ketamine is prepared which includes an excipient mixture to form an adhesive formulation in accordance with embodiments of the present invention. The solidifying formulation is prepared from the ingredients as shown in Table 23.

TABLE 23
Amitriptyline and Amitriptyline/Ketamine
formulation components.

Example

| Ingredients* | 40 | 41 | 42 | 43 |
|---|---|---|---|---|
| Isopropanol | 50.3 | 48.6 | 50.8 | 49.8 |
| Water | 2.7 | 2.6 | 2.7 | 2.7 |
| Isostearic Acid | 6.2 | 6.1 | 6.3 | 6.2 |
| Triisopropanolamine | 7.5 | 7.3 | 7.5 | 7.4 |
| Triacetin | 2.9 | 2.8 | 2.9 | 2.8 |
| Span 20 | 5.7 | 5.5 | 5.8 | 5.6 |
| Plastoid B** | 21.7 | 21.1 | 22 | 21.5 |
| Amitriptyline | 2 | 4 | | |
| Ketamine | 1 | 2 | 2 | 4 |

*Ingredients are noted as weight percent.
**from DeGussa.

The ingredients listed above are combined according to the following procedure. The drug(s), water, and triisopropanolamine are combined in a glass jar and mixed until the drug is dissolved. Then the isostearic acid, triacetin, Span 20, and isopropanol are added to the formulation and mixed well. The polymer Plastoid B is added last and heated to about 60° C. until the Plastoid B is completely dissolved. Once the polymer solution cooled to room temperature, the formulation is stirred vigorously for 2-3 minutes.

The formulations in Table 10 are applied to HMS according to Example 1, and the flux of amitriptyline and/or ketamine was measured. The results are summarized in Table 24:

TABLE 24
Steady-state flux of Amitriptyline and Amitriptyline/Ketamine
through hairless mouse skin from various adhesive
formulations at 35° C.

| | Average | |
|---|---|---|
| | amitriptyline | Average |
| | flux | ketamine flux |
| Formulation | mcg/cm²/h* | mcg/cm²/h* |
| Example 40 | 3 ± 1 | 15 ± 4 |
| Example 41 | 7.6 ± 0.2 | 38 ± 6 |
| Example 42 | 3 ± 1 | |
| Example 43 | 8.2 ± 0.7 | |

The non-volatile solvent systems in the adhesive formulations of amitriptyline and amitriptyline/ketamine were found to exhibit the best compatibility when triacetin was used as the plastizing solvent. For example, when propylene glycol was used in place of triacetin the examples noted above the formulation turned into a soft solid in the storage container in about 12 hours. Replacing propylene glycol with trolamine resulted in a clear, flowable formulation with viscosity low enough so that is can be spread on a skin surface.

### Examples 44-47

A formulation for dermal delivery of ropivacaine is prepared which includes an excipient mixture to form an adhesive formulation in accordance with embodiments of the present invention. The solidifying formulation is prepared from the ingredients as shown in Table 25.

TABLE 25
Ropivacaine HCl formulation components.

Example

| Ingredients* | 44 | 45 | 46 | 47 |
|---|---|---|---|---|
| Ropivacaine HCl | 0.31 | 0.31 | 0.31 | 0.31 |
| Isopropanol | 2 | 2 | 2.2 | 2 |
| Water | 0.125 | 0.125 | 0.125 | 0.125 |
| Isostearic Acid | 0.36 | 0.66 | 0.41 | 0 |
| Triisopropanolamine | 0.31 | 0.34 | 0.34 | 0.34 |
| Triacetin | 0.17 | 0.19 | 0 | 0.19 |
| Span 20 | 0.34 | 0 | 0.37 | 0.66 |
| Plastoid B** | 1 | 1 | 1 | 1 |

*Ingredients are noted as parts by weight.
**from Degussa.

The ingredients listed above are combined according to the following procedure. The ropivacaine HCl, water, and triisopropanolamine are combined in a glass jar and mixed until the drug is dissolved. Then the isostearic acid, triacetin, Span 20, and isopropanol are added to the formulation and mixed well. The polymer Plastoid B is added last and heated to about 60° C. until the Plastoid B is completely dissolved. Once the polymer solution cooled to room temperature, the formulation is stirred vigorously for 2-3 minutes.

The formulations in Table 25 are applied to HMS according to Example 1, and the flux of ropivacaine was measured. The results are summarized in Table 26:

TABLE 26
Steady-state flux of Ropivacaine HCl through hairless mouse
skin from various adhesive formulations at 35° C.

| | Average flux |
|---|---|
| Formulation | mcg/cm²/h* |
| Example 44 | 56 ± 2 |
| Example 45 | 39 ± 6 |
| Example 46 | 31 ± 6 |
| Example 47 | 37 ± 9 |

The flux in each of Examples 44-47 shows the importance of the triacetin, isostearic acid, Span 20 combination in the formulation. In Examples 45-47 formulations were made without Span 20, triacetin, and isostearic acid respectively. The in vitro flux of ropivacaine was impacted. The synergistic combination of the flux-enabling non volatile solvent system is important in obtaining the maximum in vitro flux of ropivacaine.

### Example 48

This formulation has the following ingredients in the indicated weight parts:

TABLE 27

| | | Ethyl | Dermacryl | | | | |
|---|---|---|---|---|---|---|---|
| | | cellulose | 79 | | Isostearic | | |
| | | N-7 | (National | | Acid | | |
| PVA | Water | (Aqualon) | Starch) | Ethanol | (ISA) | Glycerol | Ropivacaine |
| 1 | 1.5 | 0.25 | 0.35 | 0.85 | 0.8 | 0.35 | 0.3 |

In this formulation, polyvinyl alcohol (USP grade, from Amresco) is a solidifying agent, ethyl cellulose and Dermacryl 79 are auxiliary solidifying agents. Isostearic acid and glycerol form the non-volatile solvent system while ethanol and water form the volatile solvent system. Ropivacaine is the drug.

Procedures of making the formulation:
1. Ropivacaine is mixed with ISA.
2. Ethyl cellulose and Dermacryl 79 are dissolved in ethanol.
3. PVA is dissolved in water at temperature of about 60-70 C.
4. All of the above mixtures are combined together in one container and glycerol is added and the whole mixture is mixed well.

The resulting formulation is a viscous fluid. When a layer of about 0.1 mm thick is applied on skin, a non-tacky surface is formed in less than 2 minutes.

### Examples 48-49

Anti-fungal solidifying formulations are prepared and a qualitative assessment of the solidified layer's flexibility and viscosity are evaluated. The formulation components are presented in Table 28 below.

TABLE 28

Example

| | |
|---|---|
| 48 | 49 |

| | |
|---|---|
| Components | Parts by Weight |

| | | |
|---|---|---|
| Eudragit RL-PO | 3.8 | 4.2 |
| Isostearic Acid | 2 | 2.2 |
| Ethanol | 5.3 | 3.8 |
| Neutral TE Polyol | 1 | 1 |
| Econazole | 0.09 | 0.1 |

The solidifying formulation in Example 48 has a low viscosity that was lower than may be desirable for application on a nail or skin surface. The time to form a solidified layer with this formulation is longer than the desired drying time. The formulation in Example 49 had an increase in the amount of solidifying agent (Eudgragit RL-PO) and decrease in amount of ethanol, which improves the viscosity and drying time. Example 49 has a viscosity suitable for application and an improved drying time.

### Example 50

A solidifying formulation was prepared in accordance with Table 29, as follows:

TABLE 29
Solidifying formulation for sex steroids

| Ingredient | % by weight |
|---|---|
| Ethanol | 43 |
| Water | 22 |
| Polyvinyl Alcohol | 14 |
| Glycerol | 14 |
| Polyethylene | 6 |
| Glycol | |
| Testosterone | 1 |

The ingredients of Table 29 were combined as follows:
The solidifying agent is dissolved in the volatile solvent (i.e. dissolve polyvinyl alcohol in water).
The flux enabling non-volatile solvent is mixed with the solidifying agent/volatile solvent mixture.
The resulting solution is vigorously mixed well for several minutes.
Drug is then added and the solidifying formulation is mixed again for several minutes.

### Example 51

The formulation prepared in Example 50 was tested for Skin Flux, as set forth in Table 30 below.

TABLE 30
Peel-forming formulation for sex steroids

| | Skin Flux* |
|---|---|
| System | (mcg/cm²/h) |
| Example 50 | 4 ± 1 |
| AndroGel | 6 ± 2 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

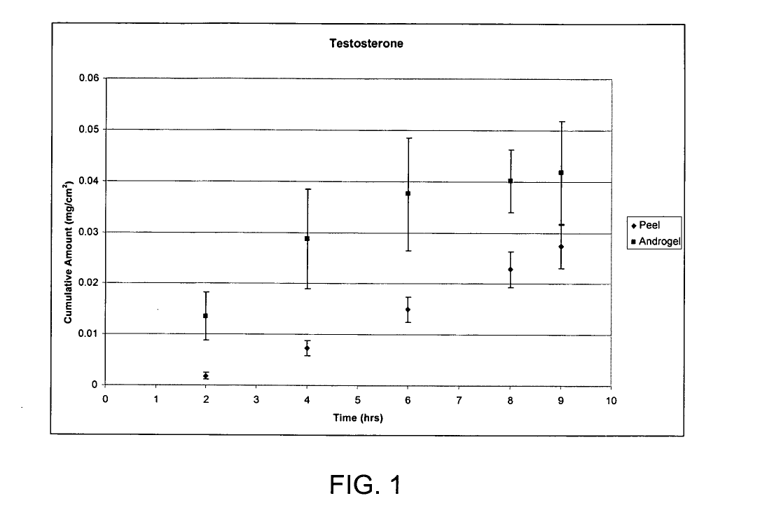

AndroGel, currently marked product, is applied directly on the hairless mouse skin and the flux determinations are made as outlined in Example 1. The steady state flux data is shown in FIG. 1. It should be noted, the steady-state flux value reported in Table 3 is determined using the linear region between 2-6 hours. As can be seen from FIG. 1, the in vitro flux of testosterone from AndroGel substantially decreases beyond 6 hours. This may be due in part to the evaporation of the volatile solvent which may act as the main vehicle for delivery. The formulation in Example 50 will deliver a steady-state amount of testosterone for at least 9 hours.

### Example 52

A stretchable adhesive formulation for transdermal delivery of ketoprofen (which is suitable for delivery via skin for treating inflammation or pain of joints and muscles) is prepared which includes saturated amount of ketoprofen in an excipient mixture (more ketoprofen than that can be dissolved in the excipient mixture) to form an adhesive formulation, some of which is prepared in accordance with embodiments of the present invention. The excipient mixture, which is a viscous and transparent fluid, is prepared using the ingredients as shown in Table 31.

TABLE 31
Ketoprofen formulation components

| | Example |
|---|---|
| Ingredients* | 52 |
| PVA (Polyvinyl Alcohol) | 10.4 |
| PEG-400 (Polyethylene Glycol) | 10.4 |
| PVP-K90 (Polyvinyl Pyrrolidone) | 10.4 |
| Glycerol | 10.4 |
| Water | 27.1 |
| Ethanol | 31.3 |
| Ketoprofen | saturated |

*Ingredients are noted as % by weight.

The compositions of Example 52 were studied for flux of ketoprofen, as shown in Table 32, as follows:

TABLE 32
Steady-state flux of Ketoprofen through hairless mouse skin
from the adhesive formulation of Example 52 at 35° C.

| | Average flux |
|---|---|
| Formulation | mcg/cm²/h* |
| Example 52 | 8 ± 3 |

*Skin flux measurement represents the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

Regarding formulation described in Example 52, ethanol and water formed the volatile solvent system, while a 1:1 mixture of glycerol and PEG 400 formed the non-volatile solvent system. Through experimentation, it is determined that PEG 400 is a slightly better solvent than glycerol for ketoprofen, while glycerol is much more compatible with PVA than PEG 400. Thus, the non-volatile solvent system of glycerol and PEG 400 are used together to provide a non-volatile solvent system for the drug, while being reasonably compatible with PVA. In additional detail with respect to the formulation in Example 65, PVA and PVP act as the solidifying agents. Further, in this embodiment, glycerol and PEG 400 also serve as plasticizers in the adhesive formulation formed after the evaporation of the volatile solvents. Without the presence of glycerol and PEG 400, a solidified layer formed by PVA and PVP alone would be rigid and non-stretchable.

### Example 53

A formulation similar to the formulation of Example 52 composition (with no ketoprofen) is applied onto a human skin surface at an elbow joint and a finger joint, resulting in a thin, transparent, flexible, and stretchable solidified layer. After a few minutes of evaporation of the volatile solvents (ethanol and water), a solidified layer is formed. The stretchable solidified layer has good adhesion to the skin and does not separate from the skin on joints when bent, and can easily be peeled away from the skin.

### Example 54

A stretchable adhesive formulation for transdermal delivery of ketoprofen (which is suitable for delivery via skin on joints and muscles) is prepared which includes saturated amount of ketoprofen in an excipient mixture (more ketoprofen than that can be dissolved in the excipient mixture) to form an adhesive formulation, some of which are prepared in accordance with embodiments of the present invention. The excipient mixture, which is a viscous and transparent fluid, is prepared using the ingredients as shown in Table 33.

TABLE 33

| | FORMULATIONS | | |
|---|---|---|---|
| Ingredients* | A | B | C |
| PVA (Celvol 502 MW 10,000) | 24.4 | | |
| PVA (Amresco MW 31,000-50,000) | | 24.4 | |
| PVA (Celvol 523 MW 125,000) | | | 41.7 |
| Water | 33.4 | 33.4 | 58.3 |
| Ethanol | 8.9 | 8.9 | |
| PG | 17.8 | 17.8 | |
| Glycerol | 11.1 | 11.1 | |
| Gantrez ES 425 | 4.4 | 4.4 | |

*Ingredients are noted in weight percent.

Formulations A and B are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.

Preparation of the PVA in water solution in Formulation C was not feasible for this molecular weight of PVA at the percentages noted. Formulation C demonstrates that the correct polymer molecular weight for PVA is important to obtain the desired formulation properties.

Formulations A and B are placed on the skin of human volunteers. After a period of several hours, long enough for the volatile solvent to evaporate, the solidified layers were removed by the volunteers and the peelability properties were evaluated. In all instances the volunteers reported that formulation example A could not be removed in one or two pieces, but was removed in numerous small pieces. Formulation example B removed in one or two pieces. The brittle nature of formulation A is attributed to the lower molecular weight PVA sample (Celvol). Low molecular weight PVA does not possess the same cohesive strength as higher molecular weight PVA material (Amresco) due to the reduced size of the polymer chain leading to a reduction in the degree of cross linking and physical interactions between individual PVA polymer chains. The reduced PVA chain interactions lead to a weakened solidified layer that is unable to withstand the mechanical forces the solidified layer is subjected to upon removal.

### Example 55-56

A stretchable adhesive formulation for transdermal delivery of ketoprofen (which is suitable for delivery via skin on joints and muscles) was evaluated which includes a placebo excipient mixture which will form an adhesive formulation, some of which are prepared in accordance with embodiments of the present invention. The excipient mixture, which is a viscous and transparent fluid, is prepared using the ingredients as shown in Table 34.

TABLE 34

Examples

| Ingredients* | 55 | 56 |
|---|---|---|
| PVA (Amresco MW 31,000-50,000) | 20.41 | 21.28 |
| Water | 30.61 | 27.66 |
| Ethanol | 20.41 | 21.28 |
| PG | 20.41 | 21.28 |
| Glycerol | 6.12 | 6.38 |
| Gantrez S97 | 2.04 | 2.13 |

*Ingredients are noted in weight percent.

Solidifying formulations in Examples 55 and 56 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez S97 is added to the mixture.
The resulting solution is vigorously mixed for several minutes.

Formulations above were applied on the forearms of study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the solidified layer from the skin or leaving residue behind. The time when this observation is made is defined as the drying time for the solidifying formulation. The results of the study are summarized in Table 35 below.

TABLE 35

| Example | Drying Time (min) |
|---|---|
| 55 | 7.0 |
| 56 | 6.5 |

The amount of water in the formulation did not significantly influence the time for the formulation to dry. However, it was noted during the study that the formulation was difficult to expel from the sample tube. After approximately 4 weeks after the formulation in Examples 55 and 56 were made the sample tubes were retrieved and were evaluated for ease of dispensing the formulation. It was noted that the formulation was impossible to expel from the tube. Interpolymer complexation between Gantrez S-97 and PVA through electrostatic interactions, hydrophobic interactions, hydrogen bonding, or Van der Waals interactions is hypothesized to be the reason(s) for the observed thickening. Moreover, the extent of this interaction may be dependent on the stoichiometric ratio of the two polymers.

### Example 57-60

A stretchable adhesive formulation for transdermal delivery of ketoprofen (which is suitable for delivery via skin on joints and muscles) was evaluated which includes an excipient mixture which will form an adhesive formulation, some of which are prepared in accordance with embodiments of the present invention. The excipient mixture, which is a viscous and transparent fluid, is prepared using the ingredients as shown in Table 36.

TABLE 36

Examples

| Ingredients* | 57 | 58 | 59 | 60 |
|---|---|---|---|---|
| PVA (Amresco MW | 22.1 | 24.4 | 22.1 | 21.1 |
| 31,000-50,000) | | | | |
| Water | 26.6 | 29.2 | 30.9 | 33.8 |
| Ethanol | 12.6 | 4.2 | 8.4 | 8.2 |
| Butanol | 0.4 | 0.5 | 0.4 | 0.4 |
| PG | 19.9 | 21.9 | 17.7 | 16.9 |
| Glycerol | 8.8 | 9.7 | 11 | 10.6 |
| Gantrez ES 425 | 4.6 | 5.1 | 4.4 | 4.0 |
| Ketoprofen | 5.0 | 5.0 | 5.1 | 5.0 |

*Ingredients are noted in weight percent.

Solidifying formulations in Examples 57-60 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.
After mixing, ketoprofen is added and the final mixture is vigorously mixed again for several minutes.

Formulations noted above were placed in laminate packaging tubes and stored at 25 C/60% RH and 40 C/75% RH conditions until pulled for testing. Physical testing was performed on each formulation. Examples 57-59 have been studied the longest and the resulting viscosity increase necessitated the desire to study the viscosity of Example 60. Table 37 summarizes the data generated on each formulation.

TABLE 37

| | Viscosity* | | | | | |
|---|---|---|---|---|---|---|
| Example | cPs | | | | | |
| Storage | | 2 | 4 | 8 | 12 | 16 |
| Cond. | T = 0 | weeks | weeks | weeks | weeks | weeks |
| 57 | 96000 | 670000 | >2500000 | Not | | |
| 25 C./ | | | | measured | | |
| 60% RH | | | | | | |
| 57 | 96000 | 500000 | 587500 | 2320000 | | |
| 40 C./ | | | | | | |
| 75% RH | | | | | | |
| 58 | 168500 | 204500 | 251000 | >2500000 | | |
| 25 C./ | | | | | | |
| 60% RH | | | | | | |
| 58 | 168500 | 215000 | 217500 | >2500000 | | |
| 40 C./ | | | | | | |
| 75% RH | | | | | | |
| 59 | 23000 | — | 25000 | 36250 | 76250 | 57500 |
| 25 C./ | | | | | | |
| 60% RH | | | | | | |
| 59 | 23000 | — | 31000 | 40000 | 243500 | 164500 |
| 40 C./ | | | | | | |
| 75% RH | | | | | | |
| 60 | 11250 | 13750 | | | | |
| 25 C./ | | | | | | |
| 60% RH | | | | | | |
| 60 | 11250 | 17500 | | | | |
| 40 C./ | | | | | | |
| 75% RH | | | | | | |

*Viscosity measured using a RVDV 1+ viscometer at 0.5 rpm.

Examples 57 and 58 had the lowest water content of the four formulations and within 4 weeks of storage attained high viscosity values. The only difference between Examples 57 and 58 is the amount of ethanol in the formulations. It was hypothesized that reducing the level of ethanol may reduce the physical thickening of the formulation due to an incompatibility between the PVA and ethanol. The viscosity data show that the higher ethanol formulation (Example 57) had lower initial viscosity, but over the 4 weeks storage the viscosity of both Examples 57 and 58 attained viscosity values that were too high for a viable formulation. Another hypothesis for the formulation thickening is that PVA is not compatible in high concentrations when dissolved in water. Additional formulations with higher water content were prepared to determine if an optimal water amount would keep the formulation from thickening up over time. Example 59 viscosity after 16 weeks has not reached the viscosity values of the initial viscosity values of Examples 57 and 58.

Placebo versions of the formulations above were applied on study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the solidified layer or leaving residue behind. The results of the study are summarized in Table 38 below.

TABLE 38

| Example | Drying Time (min)* |
|---|---|
| 57 | 4 min 49 sec |
| 58 | 5 min 41 sec |
| 59 | 4 min 27 sec |
| 60 | 5 min 1 sec |

*average dry time value from 12 study subjects.

The presence of ethanol as a second volatile solvent appears to significantly reduce the time to dry. In data not shown a local anesthetic formulation containing only water as the volatile solvent and a ratio of water to PVA of 2:1 has a drying time of >15 minutes. Optimizing the ratio and the presence of an additional volatile solvent in formulations containing water significantly reduce the drying time. It is hypothesized that the additional volatile solvent, in this case ethanol, will hydrogen bond with the water and water will escape with the ethanol when evaporating off the skin thereby forming a solidified layer.

### Example 61-62

A stretchable adhesive formulation for transdermal delivery of ketoprofen (which is suitable for delivery via skin for treating inflammation or pain of joints and muscles) is prepared which includes ketoprofen in an excipient mixture to form an adhesive formulation, some of which is prepared in accordance with embodiments of the present invention. The solidifying formulation is prepared from the ingredients as shown in Table 39.

TABLE 39
Ketoprofen solidifying formulation components

| | Example | Example |
|---|---|---|
| Ingredients* | 61 | 62 |
| PVA | 22.1 | 18.9 |
| Water | 30.9 | 37.9 |
| Fumed Silica | | 3.0 |
| Glycerol | 11.1 | 9.5 |
| Propylene glycol | 17.7 | 15.2 |
| Gantrez ES-425 | 4.4 | 3.8 |
| Ethanol | 8.8 | 7.6 |
| Ketoprofen | 5.0 | 4.2 |

*Ingredients are noted as weight percent.

TABLE 40
Steady-state flux of ketoprofen through hairless mouse skin
from an adhesive solidifying formulations at 35° C.

| | Average flux |
|---|---|
| Formulation | mcg/cm²/h* |
| Example 61 | 25 ± 6 |
| Example 62 | 27 ± 2 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

### Example 63-65

Placebo formulations containing Gantrez ES 425 as an adhesive polymer were prepared for wear studies by volunteers. The formulations are shown as examples in Table 41. All the formulations have polyvinyl alcohol as a solidifying agent to provide tensile strength to the solidifying formulation. The amount of propylene glycol in the formulations was decreased from 19.6% (w/w) to 8.7% (w/w), and the amount of glycerol was increased by the same amount to keep the total non-volatile ratio constant. Keeping the non-volatile ratio constant is important as it determines the drying time and the duration of delivery. The placebo formulations are worn on the palms of hand and percentage adherence of the solidified layer formed after evaporation of volatile solvents was observed after 5-6 hours.

TABLE 41
Placebo formulations (% w/w ingredients)

| | Example | Example | Example |
|---|---|---|---|
| Ingredient | 63 | 54 | 65 |
| Polyvinyl Alcohol | 21.7% | 21.7% | 21.7% |
| Water | 32.6% | 32.6% | 32.6% |
| Glycerol | 8.7% | 13.0% | 19.6% |
| Propylene Glycol | 19.6% | 15.2% | 8.7% |
| Gantrez ES 425 | 4.3% | 4.3% | 4.3% |
| Oleic acid | 4.3% | 4.3% | 4.3% |
| Ethanol | 8.7% | 8.7% | 8.7% |

Wear study results on 3 volunteers show that 70-80% of solidified layer as described in Example 63 stayed on palms after a duration of 5-6 hours. However, greater than 90% of solidified layer as shown in Example 65 stayed on palms of the volunteers. These examples demonstrate that glycerol is a better plasticizer that propylene glycol for the polyvinyl alcohol polymer. It also shows that the ratio of non-volatile solvent is critical in selecting the formulation for treatment of hand dermatitis.

### Examples 66-67

Adhesive formulations containing 0.05% (w/w) clobetasol propionate and 0.15% (w/w) clobetasol propionate with polyvinyl alcohol as solidifying polymer are prepared for in-vitro flux evaluation. Propylene glycol and oleic acid are the non volatile solvents selected for facilitation of clobetasol propionate delivery. As shown in Example 65, glycerol is added as the non volatile solvent for its plasticizing properties. Ratio's of ingredients used in the two formulations are shown in Table 42.

TABLE 42
Clobetasol Propionate solidifying formulations*

| | Example | Example |
|---|---|---|
| Ingredient | 66 | 67 |
| Polyvinyl Alcohol | 22.7% | 22.7% |
| Water | 34.1% | 34.0% |
| Glycerol | 17.3% | 17.2% |
| Propylene Glycol | 7.7% | 7.7% |
| Gantrez ES 425 | 4.5% | 4.5% |
| Oleic acid | 4.5% | 4.5% |
| Ethanol | 9.1% | 9.1% |
| Clobetasol Propionate | 0.05% | 0.15% |

*Numbers do not add to 100% because of rounding in the second decimal.

Both of the compositions shown above are studied for flux of clobetasol propionate on cadaver skin from three donors. The permeation results are as shown in Table 43. Commercial clobetasol ointment (0.05% w/w) was used as a control formulation.

TABLE 43
Steady state flux of clobetasol propionate
through human cadaver skin at 35° C.

| | Control | | Example 67 |
|---|---|---|---|
| | J* (ng/ | Example 66 | J* (ng/ |
| Skin Donor | cm²/h) | J* (ng/cm²/h) | cm²/h) |
| Donor 1 | 22.4 ± 2.1 | 8.8 ± 1.9 | 29.2 ± 8.2 |
| Donor 2 | 20.0 ± 2.5 | 7.6 ± 2.5 | 18.5 ± 6.4 |
| Donor 3 | 35.0 ± 4.7 | 19.3 ± 5.9 | 24.8 ± 7.7 |
| Mean +/− SD (n = 3 | 25.8 ± 7.5 | 11.9 ± 6.5 | 24.2 ± 8.0 |
| donors) | | | |

*Skin flux measurements represent the mean and standard deviation of three determinations. Flux measurements reported are determined from the linear region of the cumulative amount versus time plots. The linear region are observed to be between 6-28 hours. If the experiment is continued, it is anticipated the steady state would continue.

As seen from Table 43 formulation described in Example 66 that contained polyvinyl alcohol as a solidifying agent and 0.05% clobetasol propionate had 46% flux of clobetasol propionate when compared to the control formulation. Increasing the clobetasol propionate concentration drug concentration to 0.15% (w/w) increased the steady state flux and the flux values were 94% of the control formulation. It is expected that longer duration of application with the solidifying formulation would increase cumulative delivery in-vivo resulting in effective treatment of dermatitis.

### Example 68

Adhesive formulations containing 0.05% (w/w) clobetasol propionate with gelatin as solidifying agent are prepared for in-vitro flux evaluation. Propylene glycol, isostearic acid, and oleic acid are used as non-volatile solvents to facilitate delivery of clobetasol. Talc is added as a filler to reduce the drying time the formulation. Ratio of ingredients used in the formulation is shown in Table 44.

TABLE 44
Clobetasol Propionate formulations*

| | Example |
|---|---|
| Ingredient | 68 |
| Fish Gelatin | 29.4% |
| Water | 22.0% |
| Ethanol | 14.7% |
| Propylene Glycol | 17.6% |
| Isostearic acid | 2.2% |
| Oleic acid | 2.2% |
| Talc | 11.8% |
| Clobetasol Propionate | 0.05% |

*Numbers do not add to 100% because of rounding in the second decimal.

Unlike the polyvinyl based formulations shown in previous examples, the fish gelatin based formulation shown in Example 44 is a water washable formulation and can be easily removed by subjects suffering from hand dermatitis. Steady state flux across human cadaver skin from 3 donors with formulation as described in Example 16 is compared to the commercial clobetasol ointment. The permeation results are shown in Table 45.

TABLE 45
Steady state flux of clobetasol propionate
through human cadaver skin at 35° C.

| | Control | Example 68 |
|---|---|---|
| | J* | J* |
| Skin Donor | (ng/cm²/h) | (ng/cm²/h) |
| Donor 1 | 39.2 ± 9.2 | 46.1 ± 14.3 |
| Donor 2 | 35.6 ± 2.1 | 52.9 ± 22.3 |
| Donor 3 | 35.6 ± 5.7 | 79.7 ± 18.4 |
| Mean +/− SD (n = 3 | 36.8 ± 5.8 | 59.6 ± 22.3 |
| donors) | | |

*Skin flux measurements represent the mean and standard deviation of three determinations. Flux measurements reported are determined from the linear region of the cumulative amount versus time plots. The linear region are observed to be between 6-28 hours. If the experiment is continued, it is anticipated the steady state would continue.

As seen from Table 45, formulation described in Example 68 has 62% higher steady state flux when compared to the commercial ointment. Higher steady state flux would result is expected to reduce inflammation in difficult to treat dermatitis and psoriasis cases.

### Example 69

Adhesive formulations containing 0.05% (w/w) clobetasol propionate with gelatin as solidifying polymer are prepared for in-vitro flux evaluation. Propylene glycol, and isostearic acid are used as non-volatile solvents to facilitate delivery of clobetasol. Fumed silica is added as a filler to reduce the drying time the formulation. Ratio of ingredients used in the formulation is shown in Table 46.

TABLE 46
Clobetasol Propionate formulations*

| Ingredient | Example 69 |
|---|---|
| Fish Gelatin | 32.2% |
| Water | 24.2% |
| Ethanol | 16.1% |
| Propylene Glycol | 19.3% |
| Isostearic acid | 4.8% |
| Fumed Silica | 3.2% |
| Clobetasol Propionate | 0.05% |

*Numbers do not add to 100% because of rounding in the second decimal.

The fish gelatin based formulation shown in Example 69 is a water washable formulation and can be easily removed by subjects suffering from hand dermatitis. Steady state flux across human cadaver skin from 4 donors with formulation as described in Example 69 is compared to the commercial clobetasol ointment. The permeation results are shown in Table 47.

TABLE 47
Steady state flux of clobetasol propionate
through human cadaver skin at 35° C.

| | Control | Example 69 |
|---|---|---|
| Skin Donor | J* (ng/cm²/h) | J* (ng/cm²/h) |
| Donor 1 | 28.2 ± 7.8 | 20.7 ± 12.8 |
| Donor 2 | 30.1 ± 14.9 | 30.6 ± 13.8 |
| Donor 3 | 36.2 ± 6.2 | 93.4 ± 7.5 |
| Donor 4 | 33.6 ± 3.9 | 101.4 ± 8.5 |
| Mean +/− SD (n = 3 donors) | 32.0 ± 8.5 | 61.5 ± 38.9 |

*Skin flux measurements represent the mean and standard deviation of three determinations. Flux measurements reported are determined from the linear region of the cumulative amount versus time plots. The linear region are observed to be between 6-28 hours. If the experiment is continued, it is anticipated the steady state would continue.

As seen from Table 47, on an average, formulation described in Example 69 has at-least similar or better steady state flux when to compared to the steady state flux with the commercial ointment. Unlike talc used in Example 68, fumed silica had a low density and is expected to have a less potential to separate from the formulation.

### Examples 70-72

Solidifying formulations for dermal delivery of ropivacaine HCl are prepared which include excipient mixtures in accordance with embodiments of the present invention. The formulations are prepared from the ingredients as shown in Table 48.

TABLE 48
Ropivacaine HCl solidifying formulation components.

| | Example | | |
|---|---|---|---|
| Ingredients* | 70 | 71 | 72 |
| Ropivacaine HCl | 6.9 | 6.5 | 6.6 |
| Isopropanol | 50.7 | 45.8 | 45.9 |
| Water | 5.5 | 5.2 | 5.2 |
| Isostearic Acid | 6.3 | 6.6 | 6.6 |
| Triethylamine | | | 3.0 |
| Diisopropanolamine | | 3.9 | |
| Cetyl alcohol | | 3.3 | 3.9 |
| Triacetin | 2.9 | 2.6 | 2.6 |
| Span 20 | 5.8 | 5.2 | 5.2 |
| Plastoid B** | 21.9 | 20.9 | 21.0 |

*Ingredients are noted as weight percent.
**from Degussa.

The ingredients listed above are combined according to the following procedure. The ropivacaine HCl, water, and the amine base (triethylamine or diisopropanolamine) are combined in a glass jar and mixed until the drug is dissolved. Then the isostearic acid, triacetin, Span 20, and cetyl alcohol (Examples 71 and 72) or isopropanol (Example 70) is added to the formulation and mixed well. The polymer Plastoid B is added last and heated to about 60° C. until the Plastoid B is completely dissolved. Once the polymer solution cooled to room temperature, the formulation is stirred vigorously for 2-3 minutes.

The formulations in Table 48 are applied to HMS according to Example 1, and the flux of ropivacaine was measured. The results are summarized in Table 49:

TABLE 49
Steady-state flux of ropivacaine HCl through hairless mouse skin
from various adhesive solidifying formulations at 35° C.

| | Average flux |
|---|---|
| Formulation | mcg/cm²/h* |
| 70 | 96 ± 14 |
| 71 | 61 ± 2 |
| 72 | 70 ± 7 |

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An adhesive solid formulation for dermal delivery of a drug, comprising:
a) a drug;
b) a solvent vehicle, comprising:
i) a volatile solvent system including at least one volatile solvent, and
ii) a non-volatile solvent system including at least two non-volatile solvents, and
c) a solidifying agent,
wherein the formulation has a viscosity suitable for application and adhesion to a skin surface prior to evaporation of the volatile solvent system, wherein the formulation applied to the skin surface forms a solidified layer after at least partial evaporation of the volatile solvent system, and wherein the drug continues to be dermally delivered after the volatile solvent system is at least substantially evaporated.

2. A formulation as in claim 1, wherein the non-volatile solvent system acts as a plasticizer for the solidified agent.

3. A formulation as in claim 1, wherein the formulation further comprises an additional agent that is added to increase adhesion of the formulation when applied to a skin surface.

4. A formulation as in claim 3, wherein the additional agent includes a member selected from the group consisting of copolymers of methylvinyl ether and maleic anhydride, polyethylene glycol and polyvinyl pyrrolidone, gelatin, low molecular weight polyisobutylene rubber, copolymer of acrylsan alkyl/octylacrylamido, aliphatic resins, aromatic resins, and combinations thereof.

5. A formulation as in claim 1, wherein the volatile solvent system comprises water.

6. A formulation as in claim 1, wherein the solvent vehicle is substantially free of water.

7. A formulation as in claim 1, wherein the volatile solvent system includes at least one solvent more volatile than water, and includes at least one member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

10. A formulation as in claim 1, wherein the volatile solvent system includes at least one solvent more volatile than water, and includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

11. A formulation as in claim 1, wherein the volatile solvent system includes at least one member selected from the group consisting of ethanol, iso-propyl alcohol, and combinations thereof.

12. A formulation as in claim 1, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

13. A formulation as in claim 1, wherein the non-volatile solvent system includes at least one member selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

14. A formulation as in claim 1, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

15. A formulation as in claim 1, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

16. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof.

17. A formulation as in claim 1, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

18. A formulation as in claim 1, wherein the drug includes multiple pharmaceutically active agents.

19. A formulation as in claim 1, wherein the drug includes at least one member selected from the group consisting of acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, and combinations thereof.

20. A formulation as in claim 1, wherein the drug includes at least one member selected from the group consisting of amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, erythromycin, clindamycin, tetracycline, bacitracin, neomycin, mupirocin, polymyxin B, quinolones, ciproflaxin, bupivacaine, alpha-2 agonists, clonidine, amitriptyline, carbamazepine, alprazolam, ketamine, ketanserin, betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, piroxicam, diclofenac, indomethacin, imiquimod, rosiquimod, salicylic acid, alpha hydroxy acids, sulfur, rescorcinol, urea, benzoyl peroxide, allantoin, tretinoin, trichloroacetic acid, lactic acid, benzoic acid, progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, testosterone, methyl testosterone, oxandrolone, androstenedione, dihydrotestosterone, estradiol, ethniyl estradiol, estiol, estrone, conjugated estrogens, esterified estrogens, estropipate, and combinations thereof.

21. A formulation as in claim 1, wherein the solidified layer is sufficiently flexible and adhesive to the skin such that when applied to the skin at a human joint, the solidified layer will remain substantially intact on the skin upon bending of the joint.

22. A formulation as in claim 1, wherein the solidified layer is sufficiently flexible and adhesive to the skin such that when applied to a curved skin surface or weight bearing surface on the body, the solidified layer will remain substantially intact on the skin upon bending or stretching of the skin surface or weight bearing surface.

23. A formulation as in claim 1, wherein the formulation is formulated to deliver the drug at a therapeutically effective rate for at least about 2 hours following the formation of the solidified layer.

24. A formulation as in claim 1, wherein the formulation is formulated to deliver the drug at a therapeutically effective rate for at least about 4 hours following the formation of the solidified layer.

25. A formulation as in claim 1, wherein the formulation is formulated to deliver the drug at a therapeutically effective rate for at least about 8 hours following the formation of the solidified layer.

26. A formulation as in claim 1, wherein the formulation is formulated to deliver the drug at a therapeutically effective rate for at least about 12 hours following the formation of the solidified layer.

27. A formulation as in claim 1, wherein the weight ratio of the non-volatile solvent system to the solidifying agent is from about 0.1:1 to about 10:1.

28. A formulation as in claim 1, wherein the weight ratio of the non-volatile solvent system to the solidifying agent is from about 0.5:1 to about 2:1.

29. A formulation as in claim 1, wherein the volatile solvent system or the non-volatile solvent system is capable of causing human skin irritation and at least one of the at least two non-volatile solvents of the non-volatile solvent system reduces skin irritation.

30. A formulation as in claim 29, wherein the non-volatile solvent capable of reducing skin irritation includes a member selected from the group consisting of glycerin, propylene glycol, honey, and combinations thereof.

31. A formulation as in claim 1, wherein the solidified layer is formed within about 15 minutes of application to the skin surface under standard skin and ambient conditions.

32. A formulation as in claim 1, wherein the solidified layer is formed within about 5 minutes of the application to the skin surface under standard skin and ambient conditions.

33. A formulation as in claim 1, wherein the formulation has an initial viscosity prior to skin application from about 100 cP to about 3,000,000 cP.

34. A formulation as in claim 1, wherein the formulation has an initial viscosity prior to skin application from about 1,000 cP to about 1,000,000 cP.

35. A formulation as in claim 1, wherein the weight percentage of the volatile solvent system is from about 10 wt % to about 85 wt %.

36. A formulation as in claim 1, wherein the weight percentage of the volatile solvent system is from about 20 wt % to about 50 wt %.

37. A formulation as in claim 1, wherein at least one of the at least two non-volatile solvents is included to improve compatibility with the solidifying agent.

38. A formulation as in claim 1, wherein the non-volatile solvent system is capable of generating higher flux than any single non-volatile solvent in the non-volatile solvent system alone.

39. A formulation as in claim 1, wherein the non-volatile solvent system provides better plasticizing effect to the solidifying agent than any single non-volatile solvent in the non-volatile solvent system alone.

40. A formulation as in claim 1, wherein the solidified layer is coherent, flexible, and continuous.

41. A formulation as in claim 1, wherein the solidified layer, upon formation, is a soft, coherent solid that is peelable from a skin surface as a single piece or as only a few large pieces relative to the application size.

42. A formulation as in claim 1, wherein the non-volatile solvent system has better compatibility with the solidifying agent than any single non-volatile solvent in the non-volatile solvent system alone.

43. A formulation as in claim 1, wherein the solidified layer delivers the drug transdermally.

44. A method of dermally delivering a drug, comprising:
a) applying an adhesive formulation as a layer to a skin surface of a subject, the adhesive formulation, comprising:
i) a drug,
ii) a solvent vehicle, comprising:
a volatile solvent system including at least one volatile solvent, and
a non-volatile solvent system including at least two non-volatile solvents, wherein the non-volatile solvent system facilitates dermal delivery of the drug at a therapeutically effective rate over a sustained period of time, and
iii) a solidifying agent,
wherein the formulation has a viscosity suitable for application and adhesion to the skin surface prior to evaporation of the volatile solvent system;
b) solidifying the formulation to form a solidified layer on the skin surface by at least partial evaporation of the volatile solvent system; and
c) dermally delivering the drug from the solidified layer to the skin surface at a therapeutically effective rate over a sustained period of time.

45. A method as in claim 44, wherein the step of applying includes applying the adhesive solidifying formulation at a thickness from about 0.01 mm to about 3 mm.

46. A method as in claim 44, wherein the step of applying includes applying the adhesive formulation at a thickness from about 0.05 mm to about 1 mm.

47. A method as in claim 44, wherein the volatile solvent system comprises water.

48. A method as in claim 44, wherein the volatile solvent system includes at least one member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

49. A method as in claim 44, wherein the volatile solvent system includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

50. A method as in claim 44, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

51. A method as in claim 44, wherein the non-volatile solvent system includes at least one member selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

52. A method as in claim 44, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

53. A method as in claim 44, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

54. A method as in claim 44, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof.

55. A method as in claim 44, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

56. A method as in claim 44, wherein the drug includes multiple pharmaceutically active agents.

57. A method as in claim 44, wherein the drug includes at least one member selected from the group consisting of acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, and combinations thereof.

58. A method as in claim 44, wherein the solidified layer is sufficiently flexible and adhesive to the skin such that when applied to the skin at a human joint, the solidified layer will remain substantially intact on the skin upon bending of the joint.

59. A method as in claim 44, wherein the solidified layer is sufficiently flexible and adhesive to the skin such that when applied to a curved skin surface or weight bearing surface on the body, the solidified layer will remain substantially intact on the skin upon bending or stretching.

60. A method as in claim 44, wherein the solidified layer is kept on the skin for at least about 2 hours.

61. A method as in claim 44, wherein the solidified layer is kept on the skin for at least about 4 hours.

62. A method as in claim 44, wherein the solidified layer is kept on the skin for at least about 8 hours.

63. A method as in claim 44, wherein the solidified layer is kept on the skin for at least about 12 hours.

64. A method as in claim 44, wherein the solidifying agent is dispersed or solvated in the solvent vehicle.

65. A method as in claim 44, wherein the weight ratio of the non-volatile solvent system to the solidifying agent is from about 0.2:1 to about 5:1.

66. A method as in claim 44, wherein the volatile solvent or the non-volatile solvent system is capable of causing human skin irritation and at least one non-volatile solvent of the non-volatile solvent system is capable of reducing the skin irritation.

67. A method as in claim 44, wherein the solidified layer is formed within about 15 minutes of application to the skin surface under standard skin and ambient conditions.

68. A method as in claim 44, wherein the solidified layer is formed within about 5 minutes of application to the skin surface under standard skin and ambient conditions.

69. A method as in claim 44, wherein the formulation has an initial viscosity prior to skin application from about 100 cP to about 3,000,000 cP.

70. A method as in claim 44, wherein the weight percentage of the volatile solvent system is from about 10 wt % to about 85 wt %.

71. A method as in claim 44, wherein at least one of the non-volatile solvents is included to improve compatibility with the solidifying agent.

72. A method as in claim 44, wherein at least two non-volatile solvents are included to improve compatibility with the solidifying agent.

73. A method as in claim 44, wherein the solidified layer is coherent, flexible, and continuous.

74. A method as in claim 44, wherein the solidified layer, upon formation, is a soft, coherent solid that is peelable from a skin surface as a single piece or as only a few large pieces relative to the application size.

75. A method as in claim 44, further comprising the step of peeling the solidified layer from the skin after the sustained period of time to remove the solidified layer.

76. A method as in claim 44, further comprising the step of washing the solidified layer form the skin using a solvent after the sustained period of time to remove the solidified layer.

77. A solidified layer for delivering a drug, comprising:
a) a drug;
b) a non-volatile solvent system including at least two non-volatile solvents, wherein the non-volatile solvent system is capable of facilitating the delivery of the drug at therapeutically effective rates over a sustained period of time; and
c) a solidifying agent,
wherein the solidified layer is stretchable by 5% in one direction without cracking, breaking, or separating from a skin surface to which the layer is applied.

78. A solidified layer as in claim 77, wherein at least one of the non-volatile solvents in the non-volatile solvent system acts as a plasticizer for the solidifying agent.

79. A solidified layer as in claim 77, wherein solidified layer is sufficiently adhesive and flexible to remain substantially intact on a skin surface adjacent to a joint or muscle group where regular skin stretching occurs.

80. A solidified layer as in claim 77, wherein the weight ratio of the non-volatile solvent system to the solidifying agent is from about 0.1:1 to about 10:1.

81. A solidified layer as in claim 77, wherein the solidified layer is formed within 15 minutes of the application to the skin surface under standard skin and ambient conditions.

82. A solidified layer as in claim 77, wherein the solidified layer has a thickness from about 0.01 mm to about 3 mm.

83. A solidified layer as in claim 77, wherein the non-volatile solvent system includes at least two solvents selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucosides, benzoic acid, benzyl alcohol, butyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, dibutyl subecate, diethanolamine, dietthylene glycol monoethyl ether, diglycerides, dipropylene glycol, ethylene glycol, eucalyptus oil, eugenol, fat, fatty acid (esters glycerides), fatty alcohols, liquid sugars, ginger extract, glycerin, high fructose corn syrup, hydrogenated castor oil, IPM, IP palmitate, isostearic acid, lemon oil, lime oil, limonene, milk, mineral oil, monoacetin, monoglycerides, nutmeg oil, oleic acid, octyldodecanol, oleyl alcohol, olive alcohol, orange oil, palm oil, polyethylene glycol (PEG), peanut oil, PEG vegetable oil, peppermint oil, petrolatum, phenol, pine needle oil, polypropylene glycol, propylene glycol, sesame oil, spearmint oil, soybean oil, trolamine, tromethemine, vegetable oil, vegetable shortening, vinyl acetate, vitamin E, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters including PEG stearates, PEG oleates, PEG laurates, PEG fatty acid diesters including PEG dioleates, PEG distearates, PEG castor oils, glyceryl behenate, PEG glycerol fatty acid esters including PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid multisterol extract, myristyl alcohol, neutral oil, PEG octyl phenyl ethers, PEG alkyl ethers including PEG cetyl ethers, PEG stearyl ethers, PEG sorbitan fatty acid esters including PEG sorbitan diisosterates, PEG sorbitan monostearates, propylene glycol fatty acid esters including propylene glycol stearate, propylene glycol caprylate/caprate, sodium pyrrolidone carboxylate, sorbitol, squalene, stear-o-wet, triacetin, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty alcohol ethers, alkyl-amides (N,N-dimethylalkylamides), N-methyl pyrrolidone related compounds, sorbitan fatty acid surfactants including sorbitan monooleate, sorbitan trioleate, sorbitan monopalmitate, ethyl oleate, polyglycerized fatty acids, glycerol monooleate, glyceryl monomyristate, glycerol esters of fatty acids, and combinations thereof.

84. A solidified layer as in claim 77, wherein the solidifying agent includes at least one member selected from the group consisting of ammonia methacrylate, carrageenan, cellulose, cellulose derivatives including cellulose acetate phthalate aqueous, carboxymethylcellulose, methyl cellulose, hydroxyethylcellulose, ethylcellulose, hydroxypropylcellulose, cellulose acetate (microcrystalline), and cellulose polymers, carboxy polymethylene, xantham gum, divinyl benzene styrene, ethylene vinyl acetate, silicone, polyisobutylene, Shellac (FMC BioPolymer), guar gum, guar rosin, hypromellose phthalate, methyl acrylate, microcrystalline wax, polyvinyl alcohol, polyvinyl acetate, polyvinyl acetate phthalate, PVP ethyl cellulose, polyvinyl pyrrolidone (PVP), acrylate, polyethylene glycol/polyvinyl pyrrolidone copolymers, trimethyl siloxysilicate, maleic acid/anhydride copolymers, polacrilin, poloxamer, polyethylene oxide, poly glactic acid/poly-l-lactic acid, turpene resin, locust bean gum, prolamine (Zein), acrylic copolymers, polyurethane dispersions, gelatin, dextrin, starch, polyvinyl alcohol/polyethylene glycol copolymers, methyacrylic acid/ethyl acrylate copolymers, methacrylic acid and methacrylate based polymers including poly(methacrylic acid) copolymers and methylmethacrylate copolymers, esters of polyvinylmethylether/maleic anhydride copolymers, methyacrylic acid-ethyl acrylate copolymers, copolymers of methyl vinyl ether and maleic anhydride, aminoalkyl methacrylate copolymers, ammonioalkyl methacrylate copolymers, and combinations thereof.

85. A solidified layer as in claim 77, wherein the solidified layer is formulated to deliver the drug at a therapeutically effective rate for at least about 2 hours.

86. A solidified layer as in claim 77, wherein the solidified layer is formulated to deliver the drug at a therapeutically effective rate for from 2 to 12 hours.

87. A solidified layer as in claim 77, wherein the formulation is formulated to deliver the drug at a therapeutically effective rate for at least about 12 hours.

88. A solidified layer as in claim 77, wherein the solidified layer is a soft, coherent solid that is peelable from a skin surface as a single piece or as only a few large pieces relative to the application size.

89. A solidified layer as in claim 77, wherein the solidified layer is at least substantially devoid of volatile solvents, including water and any solvent less volatile than water.

90. A solidified layer as in claim 77, wherein the solidified layer is substantially devoid of water and solvents more volatile than water when the solidified layer contains no more than 10 wt % of water and solvents more volatile than water.

91. A solidified layer as in claim 77, wherein the solidified layer is substantially devoid of water and solvents more volatile than water when the solidified layer contains no more than 5 wt % of water and solvents more volatile than water.

92. A solidified layer as in claim 77, wherein the solidified layer is adhesive to the skin surface on one surface, and is non-adhesive on an opposing surface.

93. A solidified layer as in claim 77, wherein the solidified layer is flux-enabling for the drug.
